SEQUENCE LISTING (12 sequences)

SEQ ID NO: 1 - DNA, 22 bp, Artificial Sequence
  Primer
caagcagaagtgggttcaggat

SEQ ID NO: 2 - DNA, 22 bp, Artificial Sequence
  Primer
caagcagaagtgggttcaggat

SEQ ID NO: 3 - DNA, 18 bp, Artificial Sequence
  Primer
gctggctcatgcctttgc

SEQ ID NO: 4 - DNA, 22 bp, Artificial Sequence
  Primer
acctctcatgctgaagctcaca

SEQ ID NO: 5 - DNA, 20 bp, Artificial Sequence
  Primer
cagaccacgcaaggagttca

SEQ ID NO: 6 - DNA, 24 bp, Artificial Sequence
  Primer
ggcctgaaagctccctaatagact

SEQ ID NO: 7 - DNA, 20 bp, Artificial Sequence
  Primer
tcttcggagtttgggtttgc

SEQ ID NO: 8 - DNA, 20 bp, Artificial Sequence
  Primer
ttcagggcgaggaccataga

SEQ ID NO: 9 - DNA, 22 bp, Artificial Sequence
  Primer
tcctcatcgaagtgagcatctc

SEQ ID NO: 10 - DNA, 20 bp, Artificial Sequence
  Primer
cagaagcgctgcagaaacct

SEQ ID NO: 11 - DNA, 19 bp, Artificial Sequence
  Primer
gggcctatggcgaacactt

SEQ ID NO: 12 - DNA, 16 bp, Artificial Sequence
  Primer
cctccgtgggaatgct

(19) United States
(12) Patent Application Publication
(10) Pub. No.: US 2015/0166646 A1
(43) Pub. Date: Jun. 18, 2015

(54) Methods and Compositions for Treating or Diagnosing Melanoma

(71) Applicant: The Wistar Institute of Anatomy and Biology, Philadelphia, PA (US)

(72) Inventors: Tao Wang, Swarthmore, PA (US); Meenhard Herlyn, Wynnewood, PA (US); Russel E. Kaufman, Philadelphia, PA (US)

(21) Appl. No.: 14/407,607

(22) PCT Filed: Mar. 15, 2013
(86) PCT No.: PCT/US13/32413

§ 371 Date: Dec. 12, 2014

Related U.S. Application Data
(60) Provisional application No. 61/660,262, filed on Jun. 15, 2012.

(51) Int. Cl. C07K 16/24; A61K 39/395; A61K 31/437; G01N 33/574; A61K 31/4045; A61K 31/519; C12N 5/0786; C12Q 1/68; C07K 16/22; A61K 31/505

(52) U.S. Cl. CPC C07K 16/24; C07K 16/22; A61K 39/39558; A61K 31/437; A61K 31/505; A61K 31/4045; A61K 31/519; C12N 5/0645; C12Q 1/6886; G01N 33/5743; C12N 2502/30; C12Q 2600/158; C12Q 2600/16; C12Q 2600/118; G01N 2333/475; G01N 2333/7153; G01N 2333/53; G01N 2333/523; G01N 2500/04; G01N 2500/10

(57) ABSTRACT

Methods and compositions are provided for treating melanoma in a mammalian subject by reducing, inhibiting or down-regulating tumor-associated macrophage (TAM) production or activity in the subject. The methods can involve a combination of reducing macrophage production and number while administering an anti-cancer therapy. The treatment can involve the combined blocking or down-regulating of the nucleic acid or protein expression or activity, or the downstream pathway of CCL-2; with the blocking or down-regulating of the nucleic acid or protein expression or activity, or the downstream pathway of a matrix metalloprotease (e.g., MMP9). Another aspect involves blocking the expression or activity of VEGF. Another aspect involves blocking or down-regulating the expression, activity or signaling of the MAPK pathway or the PI3K-AKT-mTOR pathway.

### INCORPORATION BY REFERENCE OF MATERIAL SUBMITTED IN ELECTRONIC FORM

Applicant hereby incorporates by reference the Sequence Listing material filed in electronic form herewith. This file is labeled “WST134PCT_ST25.txt”, was created on 14 Mar. 2013, and is 2.35 KB.

### STATEMENT REGARDING FEDERALLY SPONSORED RESEARCH OR DEVELOPMENT

This invention was made with government support under Grant Nos. 5P30CA 010815-42, CA047159, CA025874, and CA114046 awarded by the National Institutes of Health. The government has certain rights in this invention.

### BACKGROUND OF THE INVENTION

Malignant melanoma is the deadliest form of skin cancer, with an overall survival rate of 25% at 1 year after diagnosis. At present melanoma is treated by surgical removal of the tumor and/or treatment with immunomodulators interferon-alpha and interleukin-2, and the chemotherapeutic agent, dacarbazine (DTIC).

The identification of specific molecular targets has opened the way for targeted therapies, yet cancer patients treated with targeted therapies relapse through acquisition of new driver mutations. The identification of driver mutations in tumor cells has led to the development of targeted therapies against molecules harboring these mutations. Thus, other treatments in clinical trials include a small molecule kinase inhibitor (Vemurafenib or Zelboraf) that specifically targets mutated BRAF (V600E mutation) which is expressed by ˜50% of melanomas and not normal tissues. This treatment is indicated for the treatment of patients with unresectable metastatic melanoma harboring this mutation. However, the targeting of mutant BRAF^{V600E} in melanoma with BRAF inhibitors (BRAFi) has demonstrated dramatic, but short-lived clinical benefits²⁻⁵.

Still other probable agents include ERK, MEK, PI3 kinase or AKT inhibitors. Still another proposed treatment targets CTLA-4, an inhibitory molecule on activated T cells. Blockade of the receptors on T cells by monoclonal antibodies, ipilimumab (directed to CTLA4) or MDX1106-01 (anti-PD1), are proposed to induce T cell activation, ultimately resulting in anti-tumor responses. See, e.g., D. Herlyn, Highlights of Novel Melanoma Therapies, 8th International Congress of the Society for Melanoma Research (SMR), Tampa, Fla., Nov. 9-11, 2011.

There remains a need in the art for effective compositions and methods for the successful early diagnosis and treatment of melanoma.

### SUMMARY OF THE INVENTION

In one aspect, a method for treating melanoma in a mammalian subject comprises reducing, inhibiting or down-regulating tumor-associated macrophage (TAM) production or activity in the microenvironment of a melanoma tumor in the subject.

In another aspect, a method for treating melanoma in a mammalian subject comprises blocking or down-regulating the nucleic acid or protein expression or activity, or the downstream pathway of CCL-2; and blocking or down-regulating the nucleic acid or protein expression or activity, or the downstream pathway of a matrix metalloprotease (e.g., MMP9). This combined therapy significantly inhibits and/or diminishes macrophage-induced melanoma invasion.

In another aspect, a method for treating melanoma in a mammalian subject involves administering an agent, e.g., a M-CSFR inhibitor that reduces, inhibits or down-regulates TAM production or activity before, simultaneously with, or after, administration to the subject of a therapeutic agent directed against the tumor.

In still another aspect, a method for treating melanoma in a mammalian subject comprises treating a subject with melanoma with a known anti-melanoma reagent, e.g., a BRAF inhibitor; and down-regulating macrophage activity in the microenvironment of the melanoma tumor before, simultaneously with, or after treatment with the anti-melanoma reagent, e.g., BRAF inhibitor. This method, particularly where the subject has BRAF mutant melanoma, improves clinical outcome in the subject.

In another aspect, a method of diagnosing melanoma or determining its clinical prognosis in a mammalian subject comprises detecting or measuring an upregulation of nucleic acid expression or activity or an increase in the protein expression or activity of one or more of the genes of Table 1 in macrophages in the subject compared to a non-disease or normal control. In one embodiment, the one or more genes include GPMNB.

In another aspect, a method of differentiating human monocytes to macrophages comprises culturing human monocytes in tumor cell derived conditioned media. In one embodiment, the MCM is supplemented with MCF-1, M-CSF, or GM-CSF plus IL-4.

Other aspects and advantages of these compositions and methods are described further in the following detailed description of the preferred embodiments thereof.

### BRIEF DESCRIPTION OF THE DRAWINGS

FIG. 1A is a bar graph plotting relative live cell number of 1205Lu melanoma cells treated with the increasing concentrations, e.g., 0 μM, 3 μM and 10 μM, of PLX4720 (C₁₇H₁₄ClF₂N₃O₃S; Selleck Chemicals) in the presence (dark bars) or absence (clear bars) of macrophages (Mφ), and stained with trypan blue as described in Example 1 below. The proportion of viable cells relative to the control was determined. The figure represents a plot of Mean±SD (n=3) of the percent of viable cells versus the DMSO control. ***, P<0.01.

FIG. 1B is a DNA tracing of the cells of FIG. 1A stained with propidium iodide (PI) and analyzed by flow cytometry. Each trace shows the proportion of cells with sub-G1 DNA content is indicated in each panel, as well as the total G0/G1 proportion. The top panels shown the cells treated with PLX4720 without Mφ, the lower panels show the same amount of PLX4720 with M.

FIG. 2 is a Western gel showing the results of treating melanoma cells (1205Lu) with PLX4720 (10 μM; 10 under the x axis) or no PLX4720 (0 under the x axis) in the presence (+ under the x axis) or absence (− under the x axis) of Mφ for 24 hours. Cell lysates were analyzed by Western blot for phospho-ERK, total ERK, pRSK90, pAKT, pS6, pRSK90, phospho-4EBP1, pCRAF, pNF-KB P65. RAb11 or HSP90 was used as a loading control. The amounts of PLX4720 and presence or absence of Mφ are shown below the gels

FIG. 3A is a Western gel showing cell lysates from melanocytes and melanoma cells was analyzed for M-CSFR, using actin as a loading control.

FIG. 3B is a bar graph showing melanoma (1205Lu) cells treated with DMSO, BRAFi alone, GW2580 alone, or BRFAFi and GW2580 (10 μM) in combination, with or without MCMI-Mφ for 3 days. Cells were, harvested, and stained with PI for flow cytometry analysis, using Rab11 as a loading control. The presence or absence of Mφ with a specified reagent is shown below the gels. The results are shown as percentage of G1 (darkest gray bars), G2 (palest gray bars), S (medium gray bars) and Sub-G1 (light gray bars).

FIG. 3C is a bar graph showing macrophages treated as in FIG. 3A, harvested, and stained with PI for flow cytometry analysis, using Rab11 as a loading control. The specified reagent is shown below the gels and the bars are coded as for FIG. 3A.

FIG. 4A show the results of FACS analysis performed for cell surface expression of the M2-MΦ surface markers (CD163 and CD206) in C8161-MΦ for monocytes from healthy donors that were cultured in the presence of GM-CSF (10 ng/ml), M-CSF (10 ng/ml), 1205Lu-MCM, or C8161-MCM for 7 days and differentiated to modified melanoma conditioned medium-induced macrophages (MCMI-MΦ), i.e., M1-MΦ, M2-MΦ, 1205Lu-MΦ, and C8161-MΦ. Gray shadow fills=isotype matched control; black lines=primary antibodies.

FIG. 4B show the results of FACS analysis performed for cell surface expression of the macrophage surface markers (CD68 and CD115) in C8161-MΦ. Gray shadow fills=isotype matched control; black lines=primary antibodies.

FIG. 4C is a FACS analysis of CD1a expression in M2-MΦ, M1-MΦ, DCs, and C8161-MΦ. Each experiment is representative of at least six independent experiments from six different healthy donors.

FIG. 4D is a bar graph showing the results of an experiment in which conditioned medium from M1-MΦ, M2-MΦ, 1205Lu-MΦ, and C8161-MΦ was harvested. Production of M2-MΦ cytokine and chemokines IL-10, CCL2, and M1-MΦcytokines IL-6 and TNFα was measured by Luminex analysis.

FIG. 4E is a graph showing the results of MCMI-MΦ inhibition of the proliferation of anti-CD3-induced proliferation of human anti-melanoma-specific T cells. Anti-melanoma-specific T-cell clones were cocultured with increased numbers of 1205Lu-MΦ in the presence of anti-CD3 (1 μg/m) for 7 days, 3H-TdR was added 16 h before the cells were harvested.

FIG. 5A shows flow cytometric analysis of the expression of CD68 in monocytes incubated in the presence of C8161-MCM with or without anti-human M-CSF (10 μg/ml) for 7 days. A slightly decreased expression of CD68 is observed in C8161-MCM.

FIG. 5B shows flow cytometric analysis of the expression of CD68 in monocytes incubated in the presence of 1205Lu MCM in the presence of anti-human M-CSF (10 μg/ml) or an isotype control antibody for 7 days. A slightly decreased expression of CD68 is observed in 1205Lu MCM.

FIG. 5C shows flow cytometric analysis of the melanoma cells from RGP (Sbc1-2, WM35, WM3211), VGP (WM98, WM164, WM793) and metastatic (451Lu, 1205Lu, C8161) melanomas seeded in 6-well plates, and incubated for 3 days. Culture media were harvested and the production of M-M-CSF was measured using Luminex analysis.

FIG. 5D shows flow cytometric analysis of the melanoma cells treated as in FIG. 5C, except that the production of LIF was measured using Luminex analysis.

FIG. 5E shows flow cytometric analysis of the melanoma cells treated as in FIG. 5C, except that the production of IL-6 was measured using Luminex analysis.

FIG. 5F shows flow cytometric analysis of the melanoma cells treated as in FIG. 5C, except that the production of VEGF was measured using Luminex analysis.

FIG. 5G shows flow cytometric analysis of the melanoma cells treated as in FIG. 5C, except that the production of CCL2 was measured using Luminex analysis.

FIG. 5H shows flow cytometric analysis of the melanoma cells treated as in FIG. 5C, except that the production of GM-CSF was measured using Luminex analysis.

FIG. 6A is a gene set enrichment analysis over 186 KEGG pathways, which with Bonferroni correction, identified 26 pathways significantly expressed under a FWER level of 0.05. Gene profiling reveals an invasive signature in MCMI-MΦ.

FIG. 6B is a bar graph showing the results of real-time PCR used to verify top up-regulated chemokines related to the invasive phenotype, CCL2, CCL8, and CXCL5. Data are representative of three independent experiments with three healthy donors.

FIG. 6C is a bar graph showing the results of Luminex analysis used to verify the expression of chemokines and cytokines related to the invasive phenotype.

FIG. 6D is a bar graph showing the results of real-time PCR used to verify the top up-regulated genes (MMP9 and MMP7) identified by microarray analysis. Data are representative of three independent experiments with three healthy donors.

FIG. 6 E is missing. Western blot results.

FIG. 7 is a bar graph summary of all data from an experiment showing the synergistic effect of blockade CCL2 and MMPs on MCMI-MΦ-induced melanoma invasion, 1205Lu melanoma cells were seeded into Matrigel precoated Transwells and were incubated for 18 h. Conditioned medium from 1205Lu-MΦ or control medium was added to the bottom chamber. Migrated cells were stained (using a Diff-Quick staining kit) and photographed. Micrographs (data not shown) was obtained for control medium, 1205Lu-MΦ CM, 1205Lu-MΦ CM+anti-CCL2 (10 μg/ml), 1205Lu-MΦ CM+MMPi (5 μM), and 1205Lu-MΦ CM+anti-CCL2+MMPi. Blockade of CCL-2 alone marginally increased melanoma cell invasion. Blockade of MMPi alone marginally increased melanoma cell invasion. Blockade of both MMPs and CCL-2 resulted in the significant inhibition of melanoma invasion. Data are representative of three independent experiments. ***P<0.01.

FIG. 8 is a bar graph showing the results of real-time PCR which revealed that the expression of the pro-invasive gene, GPMNB, is induced in MCMI-MΦ, i.e., it is up-regulated in C8161-MΦ and 1205Lu-MΦ compared with monocytes.

FIG. 9A shows two graphs of relative cell growth determined using a WST-1 assay for 1205Lu (left graph) and A375 cells (right graph) co-cultured in the presence (dashed line) or absence (dotted line) of macrophages (Mph) with indicated concentration of PLX4720 for 3 day. Relative growth was calculated as the ratio of treated cells to untreated cells (without Mph co-culture) at each dose. Data shown are mean±s.d. (n=4). ***P<0.001. Macrophages are shown to be essential for melanoma cell growth and survival under BRAF inhibition.

FIG. 9B are two sets of scatterplots for the 1205Lu cells (left scatterplots) and A375 cells (right scatterplots) treated as in FIG. 9A. Cell death was determined by flow cytometry using Annexin V and 7-AAD staining. DMSO was used as a control.

FIG. 9C are two sets of immunoblots for the 1205Lu cells (left immunoblot) and A375 cells (right immunoblot) treated as in FIG. 9A. Melanoma cells were harvested for immunoblotting of indicated antibodies. Mph are shown to activate p-ERK, but not p-AKT signaling in melanoma cells when PLX4720 was present.

FIG. 9D are two micrographs of two different magnifications from patients treated with BRAFi showing melanoma-infiltrating macrophages. All scale bars=50 μm.

FIG. 9E is a univariate Cox regression analysis showing statistically significant association between the number of melanoma-infiltrating macrophages with progression-free survival among 10 patients treated with BRAFi (p=0.046). The number of macrophages was the average number counted in 10 randomly selected microscope fields (see FIG. 9D).

FIG. 10A is a bar graph showing that macrophage-derived VEGF confers melanoma resistance to BRAFi; namely that VEGF rescues PLX4720-induced melanoma growth inhibition and cell death in the presence of PLX4720. 1205Lu and A375 cells were cultured with VEGF (10 ng/ml) in the presence of PLX4720 for 3 days. Cell growth was determined by WST-1 assay as in FIG. 9A. Data shown are mean±s.d. (n=4). **P=0.0014 for 1205Lu, P=0.004 for A375.

FIG. 10B is a set of scatterplots showing cell death determined for the cells treated as in FIG. 10. Cell death was determined by flow cytometry using Annexin V and 7-AAD staining.

FIG. 10C are two immunoblots showing that VEGF increases the activation of MAPK pathway. 1205Lu (top blot) and A375 (bottom blot) cells were cultured in the presence of VEGF (10 ng/ml) and PLX4720 (1 μM) for 4 hours. Cells were harvested for immunoblotting for indicated antibodies.

FIG. 10D is a bar graph showing that anti-VEGF antibody reverses macrophage-mediated melanoma resistance to PLX4720. 1205Lu and A375 cells were grown in the presence or absence of macrophages (Mph) with indicated concentration of PLX4720, anti-VEGF antibody (5 μg/ml), or both for 3 days. Cell growth was determined by WST-1 assay as in FIG. 9A. Data shown are mean±s.d. (n=4). ***P<0.001.

FIG. 10E are two sets of scatterplots showing cell death determined for the 1205Lu cells treated as in FIG. 10D. Anti-VEGF reversed macrophage-mediated anti-cell death effect.

FIG. 10F is an immunoblot of 1205Lu cells were treated as in FIG. 10D for 4 hours. Cells were harvested for immunoblotting for indicated antibodies. Anti-VEGF reversed macrophage-mediated activation of the MAPK pathway.

FIG. 11A is an immunoblot showing that BRAF inhibition activates MAPK pathway in macrophages. Macrophages (Mph) were treated with indicated concentration of PLX4720 for 2 hours. Cells were harvested for immunoblotting of indicated antibodies. BRAF inhibition paradoxically activates MAPK pathway to elicit potent biological responses in macrophages.

FIG. 11B is a bar graph showing that macrophages have high basal level of RAS activation. Macrophages were treated with PLX4720 for indicated time. ELISA assay was performed to determine the activation of RAS.

FIG. 11C is a bar graph showing that BRAF inhibition promotes macrophage growth. Macrophages were treated with the indicated concentration of PLX4720 for 72 hours. Cell growth was determined by WST-1 assay as in FIG. 9A. Data shown are mean±s.d. (n=4). ***P<0.001.

FIG. 11D is a set of scatterplots showing that BRAF inhibition protects macrophage from apoptosis. Macrophages were treated with 3 μM PLX4720 for 72 hours. Cell death was determined as in FIG. 9B.

FIG. 11E is an immunoblot showing that BRAF inhibition increases expression of PCNA.

FIG. 11F is an micrograph showing the results of immunohistochemistry analysis and revealing that Ki67 positive macrophages were present in BRAFi treated patient tissues. Blue arrow: Ki67 positive macrophages. All scale bars=50 μm.

FIG. 11G is a bar graph showing that the MEK inhibitor Trametinib (tra.) reverses PLX4720-induced macrophage proliferation. Macrophages were cultured in the presence of indicated concentrations of PLX4720 and Trametinib or both for 3 days. Cell growth was determined by WST-1 assay as in FIG. 11C. Data shown are mean±s.d. (n=4). *P<0.05. **P<0.01

FIG. 11H is a series of scatterplots for macrophages treated as in FIG. 11G and in which cell death was analyzed by flow cytometric analysis as in FIG. 11D.

FIG. 11I is an immunoblot showing that BRAF inhibition mediated ERK activation in macrophages is reversed by MEK inhibition. Macrophages were stimulated with 1 μM PLX4720 (PLX) or/and 0.5 μM Trametinib for 2 hours. Cell lysates were harvested for immunoblotting of indicated antibodies.

FIG. 11J is a series of flow cytometric traces showing that BRAF induced VEGF production. 1205Lu Mph were treated with PLX4720, Trametinib or both, and incubated for 4 hours. Intracellular staining was performed to measure expression of VEGF.

FIG. 11K is an immunoblot of macrophages treated as FIG. 11A. Cell lysates were harvested for immunoblotting of indicated antibodies.

FIG. 12A is a graph showing that GW2580 increases anti-tumor activity of PLX4720 on 1205Lu xenograft tumor growth. 1205Lu cells were injected s.c into both flanks of nude mice. When the average tumor volume reached approximately 100 mm³, the indicated doses of GW2580, PLX4720 or vehicle (n=10 for all groups, error bars indicate standard error) were administrated orally for 14 days. ANOVA was used to compare the differences in tumor volume. ***P<0.001.

FIG. 12B is a graph showing the results of flow cytometric analysis of the percent of F4/80 positive macrophages in the peritoneal cells harvested from mice treated with the indicated compositions under the x axis and that were euthanized on day 14. Data shown are mean±s.d. (n=5). *P=0.019, **P=0.00172.

FIG. 12C is a series of micrographs from immunohistochemistry analysis of the expression of F4/80 in tumors from FIG. 12A.

FIG. 12D is a series of micrographs from immunohistochemistry analysis of the expression of Ki6 in tumors from FIG. 12A.

FIG. 12E is a series of micrographs from immunohistochemistry analysis of the expression of phospho-ERK in tumors from FIG. 12A.

FIG. 12 F is a schematic model showing macrophages switching their roles from passenger to driver for melanoma growth and survival under BRAF inhibition. All scale bars=50 μm.

FIG. 13A is a graph showing that macrophages confer melanoma cell resistance to PLX4720 when 1205SK-MEK-28 cells were co-cultured with or without macrophages in the presence PLX4720 for 3 days at the indicated concentrations. Data shown are mean±s.d. (n=4). ***P<0.001.

FIG. 13B is a graph showing that macrophages confer melanoma cell resistance to PLX4720 when 451 Lu cells were co-cultured with or without macrophages in the presence PLX4720 for 3 days at the indicated concentrations. Data shown are mean±s.d. (n=4). ***P<0.001.

FIG. 14A is a graph showing that macrophages confer melanoma cell resistance to a BRAFi, Dabrafenib, when 1205Lu cells were co-cultured with or without macrophages in the presence of indicated concentrations of Dabrafenib for 3 days. Data shown are mean±s.d. (n=4). ***P<0.001.

FIG. 14B is a graph showing that macrophages confer melanoma cell resistance to a BRAFi, Dabrafenib, when A375 cells were co-cultured with or without macrophages in the presence of indicated concentrations of Dabrafenib for 3 days. Data shown are mean±s.d. (n=4). ***P<0.001.

FIG. 14C is a graph showing that macrophages confer melanoma cell resistance to a BRAFi, Dabrafenib, when SK-MEL-28 cells were co-cultured with or without macrophages in the presence of indicated concentrations of Dabrafenib for 3 days. Data shown are mean±s.d. (n=4). ***P<0.001.

FIG. 14D is a graph showing that macrophages confer melanoma cell resistance to a BRAFi, Dabrafenib, when 451 Lu cells were co-cultured with or without macrophages in the presence of indicated concentrations of Dabrafenib for 3 days. Data shown are mean±s.d. (n=4). ***P<0.001.

FIG. 15A is a graph showing that Trametinib reverses macrophage-mediated BRAFi resistance. 1205Lu melanoma cells were co-cultured with or without macrophages in the presence of indicated concentrations of PLX4720, Dab., Trametinib (Tra.), or combinations for 3 days. Cell growth was determined by WST-1 assay as described above. Data shown are mean±s.d. (n=4). ***P<0.001.

FIG. 15B is a graph showing that Trametinib reverses macrophage-mediated BRAFi resistance. A375 melanoma cells were co-cultured with or without macrophages in the presence of indicated concentrations of PLX4720, Dab., Trametinib (Tra.), or combinations for 3 days. Cell growth was determined by WST-1 assay as described above. Data shown are mean±s.d. (n=4). ***P<0.001.

FIG. 16A is a bar graph showing the results of 1205Lu-conditioned medium differentiated macrophages from 3 donors that were harvested, and the production of M-CSF determined by Luminex assay.

FIG. 16B is a bar graph showing the results of 1205Lu-conditioned medium differentiated macrophages from 3 donors that were harvested, and the production of VEGF determined by Luminex assay.

FIG. 16C is a bar graph showing the results of 1205Lu-conditioned medium differentiated macrophages from 3 donors that were harvested, and the production of IL-6 determined by Luminex assay.

FIG. 16D is a bar graph showing the results of 1205Lu-conditioned medium differentiated macrophages from 3 donors that were harvested, and the production of CXCL1 determined by Luminex assay.

FIG. 16E is a bar graph showing the results of 1205Lu-conditioned medium differentiated macrophages from 3 donors that were harvested, and the production of PDGF determined by Luminex assay.

FIG. 16F is a bar graph showing the results of 1205Lu-conditioned medium differentiated macrophages from 3 donors that were harvested, and the production of TNF determined by Luminex assay.

FIG. 16G is a bar graph showing the results of 1205Lu and SK-MEL-28 cells that were cultured in the presence of PLX4720 and the indicated growth factors under the x axis for 3 days. Cell growth was determined by WST-1 assay. Data shown are mean±s.d. (n=4). PLX4720 treatment of melanoma cells reduces growth factor production.

FIG. 17A is a bar graph showing the effect of VEGF on PLX4720-induced growth inhibition and cell death. SK-MEL-28 melanoma cells were incubated with VEGF (10 ng/ml) and PLX4720 (3 μM) for 3 days. Cell growth was determined by WST-1 assay. Data shown are mean±s.d. (n=4). **P<0.01.

FIG. 17B is a bar graph showing the effect of VEGF on PLX4720-induced growth inhibition and cell death. 451Lu melanoma cells were incubated with VEGF (10 ng/ml) and PLX4720 (3 μM) for 3 days. Cell growth was determined by WST-1 assay. Data shown are mean±s.d. (n=4). **P<0.01.

FIG. 18A is a bar graph showing VEGF rescue of Dabrafenib-induced growth inhibition and cell death in melanoma cells. 1205Lu melanoma cells were incubated with Dab. (3 μM) and VEGF (10 ng/ml) for 3 days. Cell growth was determined by WST-1 assay as in FIG. 1a (a). Data shown are mean±s.d. (n=4). **P<0.01.

FIG. 18B is a bar graph showing VEGF rescue of Dabrafenib-induced growth inhibition and cell death in melanoma cells. SK-MEL-28 melanoma cells were incubated with Dab. (3 μM) and VEGF (10 ng/ml) for 3 days. Cell growth was determined by WST-1 assay as in FIG. 1a (a). Data shown are mean±s.d. (n=4). **P<0.01.

FIG. 19A is a bar graph showing that blockade of VEGF signaling with VEGFr inhibitors reverse macrophage-mediated resistance of melanoma cells. 1205Lu melanoma cells were co-cultured with or without macrophages in the presence of indicated concentrations of PLX4720 or/and Lenbatinib for 3 days. Cell growth was determined by WST-1 assay. Data shown are mean±s.d. (n=4). ***P<0.001.

FIG. 19B is a bar graph showing the results of A375 melanoma cells that were co-cultured with or without macrophages in the presence of indicated concentrations of PLX4720 or/and Lenbatinib for 3 days. Cell growth was determined by WST-1 assay. Data shown are mean±s.d. (n=4). ***P<0.001.

FIG. 19C is a bar graph showing the results of 1205Lu melanoma cells were co-cultured with or without macrophages in the presence of indicated concentrations of PLX4720 or/and Brivanib Alaninate for 3 days. Cell growth was determined by WST-1 assay. Data shown are mean±s.d. (n=4). ***P<0.001.

FIG. 19D is a bar graph showing the results of A375 melanoma cells were co-cultured with or without macrophages in the presence of indicated concentrations of PLX4720 or/and Brivanib Alaninate for 3 days. Cell growth was determined by WST-1 assay. Data shown are mean±s.d. (n=4). ***P<0.001.

FIG. 20 shows the expression of VEGF receptors in melanoma cells. 1205Lu and A375 melanoma cells were stained with the indicated antibodies for flow cytometry analyses. Grey shading indicates isotype control. Dark blue line indicated antibodies.

FIG. 21A shows that BRAF inhibition elicits potent effects in macrophages. Macrophages were treated with indicated concentration of Dab. for 2 hours. Cells were harvested for immunoblotting by indicated antibodies.

FIG. 21B is a bar graph showing the results of macrophages that were treated with the indicated concentration of Dab. for 72 hours and cell growth was determined by WST-1 assay. Data shown are mean±s.d. (n=4). ***P<0.001.

FIG. 21C is a scatterplot showing how BRAF inhibition protects macrophage from cell death. Macrophages were treated with 3 μM Dab. for 72 hours. Cell death was determined as above.

FIG. 21D is a graph showing that the number of macrophages is increased in patient samples treated with Dab. and Tra. The number of macrophages was counted in 10 randomly selected microscope fields.

FIG. 21E are cytometric tracings showing results for macrophages treated with PLX4720 for 4 hours, and intracellular staining performed to measure expression of VEGF.

FIG. 21F are cytometric tracings showing results for macrophages treated as in FIG. 21E, but flow cytometric analysis was performed to detect VEGFR1 expression.

FIG. 22A is a bar graph showing the effect of combination of PLX4720 and GW2580 on melanoma growth in a xenograft model. Tumors from FIG. 12A were weighed. The average tumor weight is indicated as mean±s.d. (n=10), ***P<0.001.

FIG. 22B is a bar graph showing the number of total peritoneal macrophages determined by the numbers of peritoneal macrophages times the percent of the F4/80 population (n=5), ***P<0.001.

FIG. 22C is a bar graph showing mouse weight after treatment as in FIG. 12A (n=5).

FIG. 22D is a bar graph showing tumor tissues (n=4 each group) from FIG. 12A stained with anti-Ki67 antibody. The number of Ki67 positive cells was averaged numbers counted from six randomly selected microscope fields in each tumor sample. Data shown are mean±s.d. (n=4). * P<0.05.

FIG. 23A is a bar graph showing that targeting of macrophages with a M-CSFR inhibitor, GW2580, has marginal effect on melanoma cell growth and cell death. GW2580 reversed macrophage-mediated cell growth when melanoma cells were treated with PLX4720. 1205Lu (right graph) and A375 (left graph) melanoma cells were co-cultured with macrophages in the presence of indicated concentrations of PLX4720 and GW2580 for 3 days. Cell growth was measured by WST-1 assay. Data shown are mean±s.d. (n=4). ***P<0.001.

FIG. 23B are bar graphs showing results for 1205Lu (right) and A375 (left) cells treated with indicated concentrations of GW2580 for 72 hours. Cell growth was measured by WST-1 assay. Data shown are mean±S.D. (n=4).

### DETAILED DESCRIPTION OF THE INVENTION

This invention provides compositions, e.g., therapeutic agents, and methods for treating melanoma which involve inhibiting or down-regulating tumor-associated macrophage (TAM) production or activity in the subject systemically or in the microenvironment of a melanoma tumor in the subject. More specifically, the inventors have discovered that macrophages confer resistance to BRAF inhibitors in melanoma. The invention, in one embodiment, relates to down-regulating macrophage activity in the tumor microenvironment to improve clinical outcomes in patients with BRAF mutant melanoma being treated with BRAF inhibitors, such as PLX4720.

The compounds and methods of the present invention have applications in therapy of melanoma and possibly other proliferative diseases either alone or in combination with other therapies.

### I. DEFINITIONS AND COMPONENTS OF THE INVENTION

Technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention.

Macrophages (MΦ) are the most abundant leukocytes in melanoma lesions. Macrophages have been classified as activated macrophages (M1-MΦ and ‘alternatively activated macrophages’ (M2-MΦ, largely based on factors they produce. M1-MΦ are induced by proinflammatory factors, produce a lower level of IL-10 and high levels of IL-12, IL-6, and TNF-α and have antitumor activity. Conversely, M2-MΦ produce high levels of IL-10, TGFβ, CCL1, and CCL-22 and a lower level of IL-12 and promote tumor growth and metastasis.

Tumor-associated macrophages (TAMs) appear to be involved in every stage of melanoma progression and metastasis and provide an inflammatory microenvironment that plays essential roles in tumor progression and metastasis. TAMs are derived from blood monocytes and differentiate within the tumor microenvironment owing to factors produced by tumor cells. Experimentally, TAMs can be differentiated from peripheral blood monocytes by factors secreted from tumor cells and by stroma cells. A major factor that differentiates monocytes to TAMs is M-CSF. Other factors, such as VEGF-A, CCL2, IL-6, LIF, and GM-CSF, have also been reported to be involved in the differentiation of monocytes to macrophages. Most TAMs characterized to date demonstrate an M2-MΦ phenotype. However, current evidence suggests that TAMs are a mixed population bearing both M1 and M2 phenotypes.

Matrix metalloproteases (MMPs) are enzymes that degrade the extracellular matrix and result in tissue remodeling, invasion and metastasis. Among such known proteases, including about 28 known MMPs in humans, including the 92-kDa pro-MMP-9 zymogen, and the 82-KDa activated forms of MMP-9. The nucleic acid and protein sequences of these MMPs are publically available from NCBI database, among others.

The term “anti-melanoma reagents” include known therapeutic compositions for the treatment of melanoma including BRAF inhibitors, such as Vemurafenib or Zelboraf, or PLX4720, a 7-azaindole derivative that inhibits B-Raf^{V600E} with an IC50 of 13 nM, or inhibitors of ERK, MEK, PI3 kinase or AKT, or chemotherapeutic agents, such as dacarbazine (DTIC). Such agents may include CTLA-4 or PD-1 ligands, such as ipilimumab (directed to CTLA4) or MDX1106-01 (anti-PD1).

The term “target nucleic acid” or “target protein” as used herein means any nucleic acid sequence or protein, the expression or activity of which is to be modulated. The target nucleic acid can be DNA or RNA.

The term “target cells” as used herein refers to those cells in which the target nucleic acid or protein is to suppressed or overexpressed. In one embodiment, the target cell is tumor-associated macrophage (TAM). Other target cells will be obvious from the description below.

The term “homolog” or “homologous” as used herein with respect to any target sequence means a nucleic acid sequence or amino acid sequence having at least 35% identity with the mRNA or protein sequence, respectively, of the target sequence used for comparison and encoding a gene or protein having substantially similar function to that of the reference sequence. Such homologous sequences can be orthologs, e.g., genes in different species derived from a common ancestor. In other embodiments, the homolog can have at least 40, 50, 60%, 70%, 80%, 90% or at least 99% identity with the respective human target sequence. In one embodiment, the homolog is that of a non-human mammalian species. Based on the known and publically available sequences of these known human target sequences and the available computer programs readily available, such as the BLAST program, one of skill in the art can readily obtain full-length homologs, orthologs or suitable fragments of the target genes or proteins referred to herein from other mammalian species.

The term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). Complete or 100% complementarity refers to the situation in which each nucleotide unit of one polynucleotide strand or region can hydrogen bond with each nucleotide unit of a second polynucleotide strand or region. Complementarities less than 100%, e.g., 95%, 90%, 85%, refers to the situation in which 5%, 10% or 15% of the nucleotide bases of two strands or two regions of a stated number of nucleotides, cannot hydrogen bond with each other.

The term “gene” as used herein means a nucleic acid that encodes a RNA sequence including but not limited to structural genes encoding a polypeptide.

The term “sense region” as used herein means a nucleotide sequence of a small nucleic acid molecule having complementarity to a target nucleic acid sequence. In addition, the sense region of a small nucleic acid molecule can comprise a nucleic acid sequence having homology with a target nucleic acid sequence.

The term “antisense region” as used herein means a nucleotide sequence of a small nucleic acid molecule having a complementarity to a target nucleic acid sequence. It can also comprise a nucleic acid sequence having complementarity to a sense region of the small nucleic acid molecule.

The term “modulate” or “modulates” means that the expression of the gene or level of RNA molecule or equivalent RNA molecules encoding one or more protein or protein subunits or peptides, or the activity of one or more protein subunits or peptides, is up regulated or down regulated such that the expression, level, or activity is greater than or less than that observed in the absence of the modulator. The term “modulate” includes inhibit or over-express, depending upon the use.

As used herein, the term “subject”, “patient”, or “mammalian subject” includes primarily humans, but can also be extended to include domestic animals, such as dogs and cats, and certain valuable animals, such as horses, farm animals, laboratory animals (e.g., mice, rats, non-human primates) and the like.

As used herein, the term “antibody,” refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), diabodies, Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

“Biological sample” as used herein means any biological fluid or tissue that contains the biomarkers or target which is desired to be measured. In certain embodiments, suitable samples for use in the methods and compositions described herein are samples which require minimal invasion for testing, e.g., blood samples, including serum, plasma, whole blood, macrophages, including TAMs and non-tumor cells of the subject. It is also anticipated that other biological fluids, such as saliva or urine, vaginal or cervical secretions, and ascites fluids or peritoneal fluid may be similarly evaluated by the methods described herein. Also, circulating tumor cells or fluids containing them are also suitable samples for evaluation in certain embodiments of this invention. In other embodiments, the samples include biopsy tissue, tumor tissue, surgical tissue, circulating tumor cells, or other tissue. Such samples may further be diluted with saline, buffer or a physiologically acceptable diluent. Alternatively, such samples are concentrated by conventional means.

“Control”, “Reference subject” or “Reference Population” defines the source of the reference standard or control for use in a diagnostic method. In one embodiment, the reference is a human subject or a population of subjects having no cancer, i.e., healthy controls or negative controls. In yet another embodiment, the reference is a human subject or population of subjects with one or more clinical indicators of a selected cancer, e.g., melanoma. In still another embodiment, the reference is a human subject or a population of subjects who had an cancer or tumor, e.g., melanoma, following surgical removal thereof. In another embodiment, the reference is a human subject or a population of subjects who had a cancer, e.g., melanoma, and were evaluated for biomarker levels prior to surgical removal of the tumor or cancer cells. Similarly, in another embodiment, the control or reference is a human subject or a population of subjects evaluated for biomarker levels following therapeutic treatment for the cancer, e.g., melanoma. In still another embodiment, the reference is a human subject or a population of subjects prior to therapeutic treatment for the cancer. In still other embodiments of methods described herein, the reference or control is obtained from the same subject or patient who provided a temporally earlier biological sample. That control or reference sample can be pre- or post-therapy or pre- or post-surgery. In another embodiment, the reference standard is a combination of two or more of the above reference standards.

Selection of the particular class of reference standards, reference population, biomarker levels or profiles depends upon the use to which the diagnostic/monitoring methods and compositions are to be put by the physician and the desired result, e.g., initial diagnosis of the cancer, e.g., melanoma, clinical management of patients with the cancer after initial diagnosis, including, but not limited to, monitoring for reoccurrence of disease or monitoring remission or progression of the cancer, e.g., melanoma, and either before, during or after therapeutic or surgical intervention, selecting among therapeutic protocols for individual patients, monitoring for development of toxicity or other complications of therapy, predicting development of therapeutic resistance, and the like.

As used herein the term “pharmaceutically acceptable carrier” or “diluent” is intended to include any and all solvents, dispersion media, coatings, antibacterial and antifungal agents, isotonic and absorption delaying agents, and the like, compatible with administration to humans. In one embodiment, the diluent is saline or buffered saline.

It should be understood that while various embodiments in the specification are presented using “comprising” language, under various circumstances, a related embodiment is also intended to be described using “consisting of” or “consisting essentially of” language. It is to be noted that the term “a” or “an”, refers to one or more, for example, “an anti-tumor T cell” is understood to represent one or more anti-tumor T cells. As such, the terms “a” (or “an”), “one or more,” and “at least one” is used interchangeably herein

### II. THERAPEUTIC COMPOSITIONS

Therapeutic compositions are described for use in the methods of this invention, as further exemplified by the examples. In one embodiment a composition comprises an agent, ligand or compound that inhibits or down-regulates macrophage production or activity in the subject for use in the treatment of melanoma. In one embodiment, the macrophages being inhibited are tumor-associated macrophages (TAM). In another embodiment, the macrophages being inhibited are normal macrophages.

Such a therapeutic composition can involve a combination which further comprises an anti-melanoma therapeutic agent. In one embodiment, the combination of the macrophage inhibitor and anti-melanoma therapeutic agent produces a synergistic response in the mammalian subject or patient. In another embodiment, the combination of the macrophage inhibitor and anti-melanoma therapeutic agent produces more than an additive response in the subject or patient.

The macrophage-inhibiting agent, ligand or compound is in one embodiment, an agent that blocks or down-regulates the nucleic acid or protein expression or activity, or the downstream pathway of CCL-2. In another embodiment, the macrophage-inhibiting agent, ligand or compound is an agent that blocks or down-regulates the nucleic acid or protein expression or activity, or the downstream pathway of a matrix metalloprotease (MMP), such as MMP9. In another embodiment, the macrophage-inhibiting agent, ligand or compound is an agent that blocks or down-regulates the nucleic acid or protein expression or activity of VEGF. In another embodiment, the macrophage-inhibiting agent, ligand or compound is an agent that blocks or down-regulates the expression, activity or signaling of the MAPK pathway. In another embodiment, the macrophage-inhibiting agent, ligand or compound is an agent that blocks or down-regulates the expression, activity or signaling of the PI3K-AKT-mTOR pathway. In another embodiment, the macrophage-inhibiting agent, ligand or compound is an agent that blocks or down-regulates the expression or activity of M-CSFR kinase. In still another embodiment, the macrophage-inhibiting agent, ligand or compound is an agent that blocks a receptor on macrophages.

Many known compounds or agents meet these requirements and may be selected for use in the methods and compositions as herein described. Some exemplary agents include, without limitation, an antibody that binds CCL-2, an antibody that binds an MMP, an antibody that binds VEGF or a VEGF receptor inhibitor. Still other examples include MEK inhibitor or a M-CSFR inhibitor, such as the GW2580 used in the examples.

In one embodiment, the combination composition includes any one of the above macrophage-inhibitors with an anti-melanoma therapeutic agent, such as a BRAF inhibitor. In one embodiment, the BRAF inhibitor is PLX4720. In still another embodiment, as supported by the examples below, is a composition comprising PLX4720 and GW2580. In one embodiment, these reagents are present in amounts which provide a synergistic response in the subject.

### III. THERAPEUTIC METHODS

All of the compositions and components described above may be used in the methods described herein for treatment of melanoma.

A method for treating melanoma in a mammalian subject, particularly a human subject, involves reducing, inhibiting or down-regulating macrophage, e.g., tumor-associated macrophage (TAM), production or activity in the subject. In one embodiment, the macrophage reduction is targeted to the physical environment near the melanoma tumor in the subject, i.e., the tumor microenvironment, or systemically.

In one embodiment, the method involves reducing, inhibiting or down-regulating TAM by a combination of blocking or down-regulating the nucleic acid or protein expression or activity, or the downstream pathway of CCL-2 and blocking or down-regulating the nucleic acid or protein expression or activity, or the downstream pathway of a matrix metalloprotease (MMP). In a specific embodiment, the MMP is MMP-9. In another embodiment, the MMP is MMP-7. In other embodiments other known MMPs are targeted.

Blocking or down-regulating the nucleic acid or protein expression or activity, or the downstream pathway of CCL-2 can be accomplished in one embodiment by administering to the subject an antibody that binds CCL-2. Suitable antibodies may be generated by known methods or obtained from the publically available sequence of human or other mammalian CCL-2 or from commercial sources, such as LifeSpan BioScience, Inc., Acris Antibodies, ThermoFisher Scientific, Inc., Genway, etc. In another embodiment, blocking or down-regulating the nucleic acid or protein expression or activity, or the downstream pathway of CCL-2 can involve administering a nucleic acid construct comprising a sequence that reduces or suppresses the expression of CCL-2 in the target cells of the subject. For example, the down regulating composition can include a nucleic acid construct comprising a short nucleic acid molecule selected from the group consisting of a short hairpin RNA (shRNA), a short interfering RNA (siRNA), a double stranded RNA (dsRNA), a micro RNA, and an interfering DNA (DNAi) molecule, optionally under the control of a suitable regulatory sequence. Such CCL2 inhibitory sequences can be produced in plasmid based systems or viral vector systems, of which many are commercially available. In still another embodiment, such a therapeutic agent is a small molecule or drug that binds to CCL2 and inhibits its function.

These anti-CCL2 compositions, including one or more of antibodies, small nucleic acid molecules, viruses, plasmids or even small drug molecules designed for such blocking may be further associated with a pharmaceutically acceptable carrier for in vivo delivery.

Similarly, blocking or down-regulating the nucleic acid or protein expression or activity, or the downstream pathway of MMP comprises treating the subject with an antibody that binds an MMP, e.g., MMP-9 or MMP-7, among others. Blocking or down-regulating the nucleic acid or protein expression or activity, or the downstream pathway of the desired MMP can be accomplished in one embodiment by administering to the subject an antibody that binds the MMP, e.g., MMP-9. Suitable antibodies may be generated by known methods or obtained from the publically available sequence of human or other mammalian MMPs, e.g., MMP-9, or from commercial sources, such as LifeSpan BioScience, Inc., Acris Antibodies, ThermoFisher Scientific, Inc., Genway, etc. In another embodiment, blocking or down-regulating the nucleic acid or protein expression or activity, or the downstream pathway of MMP can involve administering a nucleic acid construct comprising a sequence that reduces or suppresses the expression of MMP in the target cells of the subject. For example, the down regulating composition can include a nucleic acid construct comprising a short nucleic acid molecule selected from the group consisting of a short hairpin RNA (shRNA), a short interfering RNA (siRNA), a double stranded RNA (dsRNA), a micro RNA, and an interfering DNA (DNAi) molecule, optionally under the control of a suitable regulatory sequence. Such MMP inhibitory sequences can be produced in plasmid based systems or viral vector systems, of which many are commercially available. In still another embodiment, such a therapeutic agent is a small molecule or drug that binds to MMP and inhibits its function.

These anti-MMP compositions, including one or more of antibodies, small nucleic acid molecules, viruses, plasmids or even small drug molecules designed for such blocking may be further associated with a pharmaceutically acceptable carrier for in vivo delivery.

Desirably, as shown in the examples below, the compositions that inhibit CCL-2 and those that inhibit MMP are delivered together or simultaneously so as to achieve their biological effects in combination in the subjects.

In another embodiment, the method involves reducing, inhibiting or down-regulating TAM by administering to the subject a composition that binds, inhibits or down-regulations M-CSFR, e.g., a M-CSFR inhibitor. M-CSFR is expressed in all stages of melanoma cells regardless of the gene mutation status, but not melanocytes. It is thus useful as a target for all melanomas. In addition to targeting TAMs, inhibition of M-CSFR also targets melanoma cells. According to the embodiments of this method, inhibition of M-CSFR has dual effects on both melanoma and macrophages. In one embodiment, the M-CSFR inhibitor is GW2580 (5-(3-methoxy-40((4-methoxybenzyl)oxy)benzyl)pyrimidine-2,4-diamine; LC Laboratories, BioVision, Inc.). Blocking or down-regulating the nucleic acid or protein expression or activity, or the downstream pathway of M-CSFR can be accomplished in one embodiment by administering to the subject an antibody that binds M-CSFR. Suitable antibodies may be generated by known methods or obtained from the publically available sequence of human or other mammalian M-CSFR or from commercial sources, such as LifeSpan BioScience, Inc., Acris Antibodies, ThermoFisher Scientific, Inc., Genway, etc. In another embodiment, blocking or down-regulating the nucleic acid or protein expression or activity, or the downstream pathway of M-CSFR can involve administering a nucleic acid construct comprising a sequence that reduces or suppresses the expression of M-CSFR in the target cells of the subject. For example, the down regulating composition can include a nucleic acid construct comprising a short nucleic acid molecule selected from the group consisting of a short hairpin RNA (shRNA), a short interfering RNA (siRNA), a double stranded RNA (dsRNA), a micro RNA, and an interfering DNA (DNAi) molecule, optionally under the control of a suitable regulatory sequence. Such M-CSFR inhibitory sequences can be produced in plasmid based systems or viral vector systems, of which many are commercially available. In still another embodiment, such a therapeutic agent is a small molecule or drug that binds to M-CSFR and inhibits its function.

These anti-M-CSFR compositions, including one or more of antibodies, small nucleic acid molecules, viruses, plasmids or even small drug molecules designed for such blocking may be further associated with a pharmaceutically acceptable carrier for in vivo delivery.

In another embodiment, the reducing, inhibiting or down-regulation of macrophage production or activity further comprises blocking or down-regulating the nucleic acid or protein expression or activity of VEGF. Blocking or down-regulating the nucleic acid or protein expression or activity, of VEGF can be accomplished in one embodiment by administering to the subject an antibody that binds VEGF or a VEGF receptor, or administering an inhibitor of the VEGF receptor. Suitable antibodies may be generated by known methods or obtained from the publically available sequence of human or other mammalian VEGF or receptor or from commercial sources, such as described above. In another embodiment, blocking or down-regulating the nucleic acid or protein expression or activity of VEGF or its receptor can involve administering a nucleic acid construct comprising a sequence that reduces or suppresses the expression of VEGR in the target cells of the subject. For example, the down regulating composition can include a nucleic acid construct comprising a short nucleic acid molecule selected from the group consisting of a short hairpin RNA (shRNA), a short interfering RNA (siRNA), a double stranded RNA (dsRNA), a micro RNA, and an interfering DNA (DNAi) molecule, optionally under the control of a suitable regulatory sequence. Such VEGF or VEGFr inhibitory sequences can be produced in plasmid based systems or viral vector systems, of which many are commercially available. In still another embodiment, such a therapeutic agent is a small molecule or drug that binds to VEGF or VEGFr and inhibits its function.

In still another embodiment, the reducing, inhibiting or down-regulation of macrophage production or activity further comprises blocking or down-regulating the expression, activity or signaling of the MAPK pathway or the PI3K-AKT pathway. In a similar manner as described above, such biological effects may be accomplished by the use of antibodies to MAPK or PI3K-AKT, or to targets in the respective pathways, by nucleic acid constructs, or by small molecule inhibitors of these targets.

In still another embodiment, the reducing, inhibiting or down-regulation of macrophage production or activity further comprises blocking a receptor on macrophages, such as those receptors and proteins expressed on the macrophages as discussed in the examples below.

In another embodiment, one or more of the above-noted compositions used to reduce, inhibit or down-regulate TAM production or activity is administered to the subject before administration to the subject of an anti-melanoma therapeutic agent directed against the tumor. By “before” means that administration of the TAM inhibitor(s) occurs any time from at least 1 minute, 10 minutes, 30 minutes, 1 hour, 24 hours, 48 hours or up to one week or more prior to administration of the anti-melamona therapeutic agent. In another embodiment, one or more of the above-noted compositions used to reduce, inhibit or down-regulate TAM production or activity is administered to the subject after administration to the subject of an anti-melanoma therapeutic agent directed against the tumor. By “after” means that administration of the TAM inhibitor(s) occurs any time from at least 1 minute, 10 minutes, 30 minutes, 1 hour, 24 hours, 48 hours or up to one week or more after administration of the anti-melamona therapeutic agent.

In yet another embodiment, one or more of the above-noted compositions used to reduce, inhibit or down-regulate TAM production or activity is administered to the subject simultaneously with administration to the subject of an anti-melanoma therapeutic agent directed against the tumor. Simultaneous administration includes administration in a single composition or in two or more separate compositions administered at about the same time to the subject.

The combination of such therapies, in certain embodiments, results in a synergistic effect on the cancer. See, for examples the results in the figures and examples relating to the combination of the small-molecule ATP-competitive inhibitor of M-CSFR kinase, GW2580 and the BRAF inhibitor PLX4720.

In one embodiment of such a method, the therapeutic agent is a BRAF inhibitor. This method is particularly useful where the subject has BRAF mutant melanoma. This treatment improves clinical outcome in the subject. In one embodiment, the BRAF inhibitor is PLX4720.

In another embodiment, the BRAF inhibitor is Vemurafenib or Zelboraf. In another embodiment, the therapeutic agent is one or more inhibitors of ERK, MEK, PI3 kinase or AKT or mTOR. In still another embodiment, the therapeutic agent is a chemotherapeutic agent, such as dacarbazine (DTIC) or other commonly used chemotherapeutic compound. Additional therapeutic agents may include CTLA-4 or PD-1 ligands, such as ipilimumab (directed to CTLA4) or MDX1106-01 (anti-PD1). In still another embodiment, the therapeutic agent may be a combination of such therapeutic agents, administered as a single composition or administered at about the same time to the subject.

In still another embodiment, a method for improving clinical outcome in a mammalian subject having BRAF mutant melanoma comprises treating a subject with melanoma with a BRAF inhibitor; and down-regulating macrophage activity in the microenvironment of the melanoma tumor before, simultaneously with, or after treatment with the BRAF inhibitor. The BRAF inhibitors and compositions for down-regulating macrophage or TAM activity are as described above.

The therapeutic compositions administered by these methods, e.g., whether antibody, virus, virus nanoparticle, nucleic acid construct alone, nanoparticle, or small molecule drug, are administered directly into the subject or into the subject's anatomy most plagued by the disease, where possible. Conventional and pharmaceutically acceptable routes of administration include, but are not limited to, intraperitoneal, intravenous, intranasal, intravenous, intramuscular, intratracheal, subcutaneous, intratumoral or intranodal administration. Still other routes include intradermal, transdermal, intramuscular, and intraarterial. The appropriate route is selected depending on a variety of considerations, including the nature of the composition, i.e., protein, virus, nucleic acid, etc., and an evaluation of the age, weight, sex and general health of the patient and the components present in the immunogenic composition, and similar factors by an attending physician. Routes of administration may be combined, if desired. In some embodiments, the administration is repeated periodically.

These therapeutic compositions may be administered to a patient, preferably suspended in a biologically compatible solution or pharmaceutically acceptable delivery vehicle. The various components of the compositions are prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose. These methods may further employ administering a nucleic acid construct with a delivery agent, such as a lipid, a cationic lipid, a phospholipid, and a liposome. Further, these methods can comprise administering to the subject another therapeutically active agent useful to treat the disease in question. In certain embodiments, the nucleic acid constructs may be in the form of oligonucleotides or in the form of a nanoparticle complexed with a polymer or other material.

Dosages of the compositions used in these methods are readily determined by one of skill in the art will depend primarily on factors such as the stage of melanoma and location of tumor being treated, the age, weight and health of the patient, and may thus vary among patients. A suitable dose of the composition(s) is formulated in a pharmaceutical composition, as described above (e.g., dissolved in about 0.1 mL to about 2 mL of a physiologically compatible carrier) and delivered by any suitable means. Dosages are typically expressed in a “unit dosage”, which is defined as dose per subject, e.g., a unit dosage of 1 mg protein/antibody. Alternatively dosages can be expressed as amount per body weight of the subject or patient, using the norm for therapeutic conversions as 80 kg body weight. For example, a 1 mg unit dose per subject is equivalent to about 12.5 μg/kg body weight. It is anticipated that therapeutically effective dosages of known compounds or molecules will be obvious to one of skill in the art based upon their known potencies. For example, a therapeutically effective adult human or veterinary dosage of an antibody, e.g., an anti-CCL2 antibody and/or MMP-9 antibody, may be a “unit dosage” of less than about 0.01 mg to 100 mg of protein/antibody. In one embodiment, the unit dosage is 0.01 mg. In another embodiment, the unit dosage is 0.1 mg. In another embodiment, the unit dosage is 1 mg. In still another embodiment, the unit dosage is 10 mg. Even higher dosages may be contemplated.

Alternatively, a therapeutically effective adult human or veterinary dosage of a viral vector or nanoparticle carrying a short nucleic acid construct is generally in the range of from about 100 μL to about 100 mL of a carrier containing concentrations of from about 1×10⁶ to about 1×10¹⁵ particles, about 1×10¹¹ to 1×10¹³ particles, or about 1×10⁹ to 1×10¹² particles virus. In another example, a dosage of adoptive T cells delivering the nucleic acid inhibiting constructs can range from about 10⁵ to about 10¹¹ cells per kilogram of body weight of the subject. In still another example, the dosages of the small molecule drugs will be with the skill of the art depending upon the drug itself and its activity and can range from microgram to milligram levels.

Determining the timing or frequency of repeated dosage administration will include an assessment of disease in response to the initial administration and is within the skill of the attending physician.

In another embodiment, the method further comprises administering to the subject along with the therapeutic agents described herein that down-regulate TAM production or activity, other adjunctive therapy directed which may include a monoclonal antibody, chemotherapy, radiation therapy, a cytokine, or a combination thereof.

### III. DIAGNOSTIC METHODS

In another aspect, a method of diagnosing melanoma or determining its clinical prognosis in a mammalian subject involves detecting or measuring an upregulation of nucleic acid expression or activity or an increase in the protein expression or activity of at least one or a combination of the genes of Table 1 or Table 2, below, in a biological sample obtained from the subject as compared the respective expression/activity level(s) in a non-diseased control. In one embodiment, the gene/protein expression/activity being measured is GPMNB in macrophages in the subject or in a biological sample obtained from the subject, as compared to a non-diseased control. In still another embodiment a combination of such genes includes CCL2, CXCL5, and/or CCL8. In still another embodiment, a combination of such genes diagnostic of melanoma include one or more of MMP-9, 7, 1, 12, and/or secreted phosphoprotein 1 (SPP1, osteopontin), cathepsin L1 (CTSL1), and urokinase (uPA). In yet another embodiment, the combination of genes includes DFNA5. In still another embodiment, the genes forming the signature are those in Table 2 below. Still other combinations of 2, 5, 10, 20, 30, 50, 75 or all 100 of the genes listed in Table 1 may form a diagnostic signature diagnostic or prognostic of a stage of melanoma. Where in the following discussion of diagnostic reagents and methods herein, GPMNB is mentioned as the diagnostic target, it is understood that GPMNB also represents any combination of the genes/targets mentioned above or in Table 1.

TABLE 1

| Gene | | Fold Change | Parametric | |
|---|---|---|---|---|
| Symbol | Description | (C8161/Mono) | P value | Invasion |
| CCL2 | Chemokine (C-C | 260.5 | 2.08E−05 | ↑ |
| | Motif) Ligand 2 | | | |
| MMP9 | Matrix | 242.9 | 6.63E−05 | ↑ |
| | Metallopeptidase 9 | | | |
| | (Gelatinase B, 92 Kda | | | |
| | Gelatinase, 92 Kda | | | |
| | Type Iv Collagenase) | | | |
| SPP1 | Secreted | 225.4 | 8.41E−06 | ↑ |
| | Phosphoprotein 1 | | | |
| | Transcript Variant 2 | | | |
| CXCL5 | Chemokine (C-X-C | 183.5 | 1.26E−05 | ↑ |
| | Motif) Ligand 5 | | | |
| GPNMB | Glycoprotein | 128.7 | 4.11E−06 | ↑ |
| | (Transmembrane) | | | |
| | Nmb Transcript | | | |
| | Variant 2 | | | |
| C15orf48 | chromosome 15 open | 128.2 | 2.01E−05 | N/A |
| | reading frame 48 | | | |
| MT1G | Metallothionein 1G | 124 | 1.54E−04 | ↑ |
| MMP7 | Matrix | 119.8 | 1.32E−04 | ↑ |
| | Metallopeptidase 7 | | | |
| | (Matrilysin, Uterine) | | | |
| CCL8 | Chemokine (C-C | 114.1 | 9.24E−04 | ↑ |
| | Motif) Ligand 8 | | | |
| CCL22 | Chemokine (C-C | 111.2 | 7.70E−05 | ↑ |
| | Motif) Ligand 22 | | | |
| IL6 | Interleukin 6 | 94.5 | 1.11E−05 | ↑ |
| | (Interferon, Beta 2) | | | |
| CCL7 | Chemokine (C-C | 76.9 | 5.37E−04 | ↑ |
| | Motif) Ligand 7 | | | |
| CCL4L2 | Chemokine (C-C Motif) | 73.8 | 8.60E−05 | ↑ |
| | Ligand 4-Like 2 | | | |
| APOE | Apolipoprotein E | 73.2 | 8.56E−04 | ↑ |
| APOC1 | Apolipoprotein C-I | 67.8 | 5.61E−04 | ↑ |
| TNFAIP6 | Tumor Necrosis | 64.2 | 4.72E−05 | ↑ |
| | Factor, Alpha-Induced | | | |
| | Protein 6 | | | |
| IL1A | Interleukin 1, Alpha | 60.6 | 6.97E−05 | ↑ |
| TM4SF19 | Predicted: | 56.5 | 9.33E−05 | N/A |
| | Transmembrane 4 L | | | |
| | Six Family Member 19, | | | |
| | Transcript Variant 3 | | | |
| A2M | Alpha-2- | 50.6 | 7.09E−05 | ↑ |
| | Macroglobulin | | | |
| NR1H3 | Nuclear Receptor | 50.2 | 1.11E−04 | ↑ |
| | Subfamily 1, Group H, | | | |
| | Member 3 | | | |
| CTSL1 | Cathepsin L1 | 48.1 | 2.80E−04 | ↑ |
| | Transcript Variant 1 | | | |
| MT1E | Metallothionein 1E | 46.3 | 1.15E−03 | ↑ |
| PLTP | Phospholipid Transfer | 42.7 | 1.08E−03 | ↑ |
| | Protein Transcript | | | |
| | Variant 1 | | | |
| SCD | Stearoyl-Coa | 42 | 1.88E−05 | ↑ |
| | Desaturase (Delta-9- | | | |
| | Desaturase) | | | |
| CCL4L1 | Chemokine (C-C | 41.6 | 4.46E−05 | ↑ |
| | Motif) Ligand 4-Like 1 | | | |
| ADAMDEC1 | Adam-Like, Decysin 1 | 40.6 | 3.00E−04 | ↑ |
| DFNA5 | Deafness, Autosomal | 38.6 | 1.90E−04 | ↑ |
| | Dominant 5 | | | |
| | Transcript Variant 1 | | | |
| MT1H | Metallothionein 1H | 36.2 | 7.29E−05 | ↑ |
| SLC39A8 | Solute Carrier Family | 35.8 | 4.74E−04 | ↑ |
| | 39 (Zinc Transporter), | | | |
| | Member 8 Transcript | | | |
| | Variant 1 | | | |
| FPR3 | Formyl Peptide | 32 | 2.33E−04 | ↑ |
| | Receptor 3 | | | |
| CCL20 | Chemokine (C-C | 31.6 | 8.97E−04 | ↑ |
| | Motif) Ligand 20 | | | |
| MT1A | Metallothionein 1A | 30.3 | 3.38E−05 | N/A |
| CXCL1 | Chemokine (C-X-C | 29.4 | 4.08E−03 | ↑ |
| | Motif) Ligand 1 | | | |
| MT2A | Metallothionein 2A | 28.8 | 3.20E−04 | ↑ |
| SLCO2B1 | Solute Carrier Organic | 27.8 | 1.85E−04 | ↑ |
| | Anion Transporter | | | |
| | Family, Member 2B1 | | | |
| ACP5 | Acid Phosphatase 5, | 27.8 | 8.85E−05 | ↑ |
| | Tartrate Resistant | | | |
| IL1F9 | Interleukin 1 Family, | 27.5 | 2.04E−04 | N/A |
| | Member 9 | | | |
| TM4SF1 | Transmembrane 4 L | 27.3 | 5.39E−04 | ↑ |
| | Six Family Member 1 | | | |
| TGM2 | Transglutaminase 2 (C | 26.4 | 1.59E−07 | ↑ |
| | Polypeptide, Protein- | | | |
| | Glutamine-Gamma- | | | |
| | Glutamyltransferase) | | | |
| | Transcript Variant 1 | | | |
| NDP | Norrie Disease | 25.2 | 1.95E−04 | ↑ |
| | (Pseudoglioma) | | | |
| PMP22 | Peripheral Myelin | 24.4 | 5.17E−05 | ↑ |
| | Protein 22 Transcript | | | |
| | Variant 2 | | | |
| ABCA1 | Atp-Binding Cassette, | 23.8 | 9.52E−03 | ↑ |
| | Sub-Family A | | | |
| | Member 1 (Abcal) | | | |
| TM7SF4 | Transmembrane 7 | 23.8 | 5.17E−05 | ↑ |
| | Superfamily Member 4 | | | |
| IL7R | Interleukin 7 Receptor | 23.8 | 5.58E−06 | N/A |
| IL1RN | Interleukin 1 Receptor | 23.6 | 1.55E−02 | N/A |
| | Antagonist Transcript | | | |
| | Variant 4 | | | |
| JAKMIP2 | Janus Kinase And | 23.1 | 1.10E−04 | N/A |
| | Microtubule | | | |
| | Interacting Protein 2 | | | |
| RSAD2 | Radical S-Adenosyl | 22.9 | 2.41E−05 | N/A |
| | Methionine Domain | | | |
| | Containing 2 | | | |
| LPL | Lipoprotein Lipase | 22.9 | 2.49E−03 | ↑ |
| NRP1 | Neuropilin 1 | 22.9 | 1.39E−03 | ↑ |
| | Transcript Variant 3 | | | |
| MARCO | Macrophage Receptor | 22.6 | 2.03E−03 | N/A |
| | With Collagenous | | | |
| | Structure | | | |
| MT1F | Metallothionein 1F | 21.7 | 5.92E−03 | N/A |
| C1QB | Complement | 21.6 | 1.66E−03 | N/A |
| | Component 1, | | | |
| | Q Subcomponent, | | | |
| | B Chain | | | |
| INDO | Indoleamine-Pyrrole | 21.4 | 4.53E−04 | ↑ |
| | 2,3 Dioxygenase | | | |
| CCL3 | Chemokine (C-C | 21 | 9.48E−03 | ↑ |
| | Motif) Ligand 3 | | | |
| GJB2 | Gap Junction Protein, | 20.9 | 1.59E−05 | ↑ |
| | Beta 2, 26 Kda | | | |
| PLAU | Plasminogen | 20.8 | 4.24E−05 | ↑ |
| | Activator, Urokinase | | | |
| CYP27B1 | Cytochrome P450, | 20.5 | 1.49E−04 | ↑ |
| | Family 27, Subfamily B, | | | |
| | Polypeptide 1 | | | |
| | Nuclear Gene Encoding | | | |
| | Mitochondrial Protein | | | |
| SLC1A3 | Solute Carrier Family 1 | 20.2 | 1.47E−04 | ↑ |
| | (Glial High Affinity | | | |
| | Glutamate Transporter), | | | |
| | Member 3 | | | |
| CXCL2 | Chemokine (C-X-C | 19.5 | 1.78E−04 | ↑ |
| | Motif) Ligand 2 | | | |
| CD209 | Cd209 Molecule | 19.5 | 3.69E−03 | N/A |
| PTGES | Prostaglandin E | 19.5 | 6.49E−05 | ↑ |
| | Synthase | | | |
| CCL3L1 | Chemokine (C-C Motif) | 18.9 | 2.04E−04 | ↑ |
| | Ligand 3-Like 1 | | | |
| MAPK13 | Mitogen-Activated | 18.6 | 1.01E−03 | ↑ |
| | Protein Kinase 13 | | | |
| ACP2 | Acid Phosphatase 2, | 18.4 | 4.60E−03 | N/A |
| | Lysosomal | | | |
| LTB4DH | leukotriene B4 12- | 18.3 | 6.26E−07 | N/A |
| | hydroxydehydrogenase | | | |
| CCR7 | Chemokine (C-C | 17.7 | 9.87E−05 | ↑ |
| | Motif) Receptor 7 | | | |
| LOC283050 | Predicted: | 17.2 | 1.27E−03 | N/A |
| | Hypothetical Protein | | | |
| | Loc283050 | | | |
| IL23A | Interleukin 23, Alpha | 16.9 | 1.85E−04 | ↑ |
| | Subunit P19 | | | |
| VSIG4 | V-Set And | 16.4 | 5.21E−03 | ↑ |
| | Immunoglobulin | | | |
| | Domain Containing 4 | | | |
| | Transcript Variant 1 | | | |
| SLC16A10 | Solute Carrier Family 16, | 16.1 | 2.07E−04 | N/A |
| | Member 10 (Aromatic | | | |
| | Amino Acid Transporter) | | | |
| STEAP3 | Steap Family Member 3 | 16.1 | 5.08E−05 | ↑ |
| | Transcript Variant 1 | | | |
| | (Function: iron | | | |
| | homeostasis) | | | |
| CSF2 | Colony Stimulating | 15.9 | 2.39E−04 | N/A |
| | Factor 2 (Granulocyte- | | | |
| | Macrophage) | | | |
| SCG5 | Secretogranin V (7B2 | 15.9 | 2.13E−04 | ↑ |
| | Protein) | | | |
| C20orf123 | chromosome 20 open | 15.5 | 1.43E−04 | N/A |
| | reading frame 123 | | | |
| CCND1 | Cyclin D1 | 15.4 | 1.37E−04 | ↑ |
| CLEC5A | C-Type Lectin Domain | 15.4 | 7.82E−04 | N/A |
| | Family 5, Member A | | | |
| HSD11B1 | Hydroxysteroid (11- | 15.3 | 4.77E−04 | N/A |
| | Beta) Dehydrogenase 1 | | | |
| | Transcript Variant 1 | | | |
| CA12 | Carbonic Anhydrase Xii | 15.2 | 9.28E−05 | ↑ |
| | Transcript Variant 1 | | | |
| GPC4 | Glypican 4 | 15.2 | 9.46E−04 | N/A |
| COLEC12 | Collectin Sub-Family | 15.2 | 3.73E−04 | N/A |
| | Member 12 | | | |
| MTE | Metallothionein E | 14.8 | 3.35E−04 | N/A |
| WBP5 | Ww Domain Binding | 13.8 | 5.42E−04 | ↑ |
| | Protein 5 Transcript | | | |
| | Variant 4 | | | |
| LGMN | Legumain Transcript | 13.7 | 1.57E−03 | ↑ |
| | Variant 2 | | | |
| BCAT1 | Branched Chain | 13.6 | 6.32E−04 | ↑ |
| | Aminotransferase 1, | | | |
| | Cytosolic | | | |
| EMP1 | Epithelial Membrane | 13.4 | 4.30E−06 | ↑ |
| | Protein 1 | | | |
| MATK | Megakaryocyte- | 13.2 | 4.12E−04 | N/A |
| | Associated Tyrosine | | | |
| | Kinase Transcript | | | |
| | Variant 2 (cancer | | | |
| | and immunology) | | | |
| GPR84 | G Protein-Coupled | 12.9 | 1.79E−03 | N/A |
| | Receptor 84 | | | |
| MAP1LC3A | Microtubule- | 12.8 | 7.98E−05 | ↑ |
| | Associated Protein 1 | | | |
| | Light Chain 3 Alpha | | | |
| | Transcript Variant 2 | | | |
| FBP1 | Fructose-1,6- | 12.2 | 6.65E−04 | ↑ |
| | Bisphosphatase 1 | | | |
| | (metabolism) | | | |
| SLAMF8 | Slam Family Member 8 | 12.2 | 9.78E−05 | N/A |
| F3 | Coagulation Factor Iii | 12.1 | 2.84E−03 | ↑ |
| | (Thromboplastin, | | | |
| | Tissue Factor) | | | |
| RASGRP3 | Ras Guanyl Releasing | 12 | 1.18E−04 | ↑ |
| | Protein 3 (Calcium | | | |
| | And Dag-Regulated) | | | |
| GSN | Gelsolin | 11.8 | 7.77E−03 | ↑ |
| | (Amyloidosis, Finnish | | | |
| | Type) Transcript | | | |
| | Variant 2 | | | |
| FAM23B | Family With Sequence | 11.8 | 6.79E−04 | N/A |
| | Similarity 23, Member B | | | |
| CRABP2 | Cellular Retinoic Acid | 11.8 | 3.95E−04 | ↑ |
| | Binding Protein 2 | | | |
| ETV5 | Ets Variant Gene 5 | 11.6 | 1.48E−04 | ↑ |
| | (Ets-Related Molecule) | | | |
| PPAP2B | Phosphatidic Acid | 11.4 | 1.66E−03 | ↑ |
| | Phosphatase Type 2B | | | |
| | Transcript Variant 1 | | | |
| ACO1 | Aconitase 1, Soluble | 11.4 | 8.09E−05 | N/A |
| IRAK2 | Interleukin-1 | 11.3 | 5.39E−04 | ↑ |
| | Receptor-Associated | | | |
| | Kinase 2 | | | |
| CD9 | Cd9 Molecule | 11.2 | 1.72E−03 | ↑ |

Note:
↑: Has been implicated to increase invasion and tumor progression.
N/A: data not available.

The expression/activity level of GPMNB is then compared with the level of expression/activity in a healthy mammalian subject. While such comparison can occur by direct comparison with the expression/activity levels in one or more healthy subjects, it is more typical for a reference average expression/activity level to be provided as a number or range. A level of expression/activity of the GPMNB in the subject's sample that is below the level of expression/activity in a healthy mammalian subject (or reference average) is an indication of a diagnosis or severity of a cancer. In one embodiment of such a method the measuring step includes measuring GPMNB as ribonucleic acid, deoxyribonucleic acid, or protein using conventional assay technologies. In another embodiment, the expression level of GPMNB is compared to that in an earlier biological sample of the same subject (or reference sample number derived from multiple patients at various stages of melanoma). A decrease in expression/activity of GPMNB over the prior or reference sample is indicative of worsening disease, while an increase in GPMNB expression/activity over the prior or reference sample is indicative of a good prognosis or treatment.

The specific methodologies that can be employed to perform the diagnostic methods described herein are conventional and may be readily selected and adapted by one of skill in the art. Methods useful in performing the diagnostic steps described herein are known and well summarized in U.S. Pat. No. 7,081,340. Such methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription polymerase chain reaction (RT-PCR) or qPCR. Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, Calif.), differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) and high coverage expression profiling (HiCEP) analysis.

In conjunction with the performance of the various diagnostic techniques described herein, another aspect of the invention is a variant of diagnostic reagents employing GPMNB. One embodiment of a diagnostic reagent comprises at least one polynucleotide immobilized on a substrate. The polynucleotide is a genomic probe that hybridizes to GPMNB. The reagent can contain additional splicing factors useful as a genetic signature of melanoma, such as the genes identified in the figures and examples herein. In one embodiment the reagent enables detection of changes in expression in at least GPMNB and one other gene from that of a reference expression profile. Differences between the expression of these factors in a subject from that of the signature profile indicate a diagnosis of, prognosis of, or stage of, melanoma.

The diagnostic compositions of the invention can be presented in the format of a microfluidics card, a microarray, a chip or chamber employs the PCR, RT-PCR or Q PCR techniques described above. In one aspect, such a format is a diagnostic assay using TAQMAN® Quantitative PCR low density arrays. When a biological sample from a selected subject is contacted with the primers and probes in the diagnostic composition, PCR amplification of genes in the gene expression profile from the subject permits detection of changes in expression in the splicing factor genes in the gene expression profile from that of a reference gene expression profile. Significant changes in the gene expression indicating a decrease in the expression level of these splicing factors from that of the reference gene expression profile can correlate with a diagnosis of or prognosis of disease.

The selection of the GPMNB polynucleotide sequences or others from the figures and examples below, their length and labels used in the composition are routine determinations made by one of skill in the art in view of the teachings herein.

Suitable diagnostic reagents and kits containing them are useful for the measurement and detection of GPMNB or other genes identified herein in the methods described herein for diagnosis/prognosis of melanoma. In such composition, the antibodies or peptides or nucleic acid sequences may be immobilized on suitable substrates, e.g., bound to an avidin-coated solid support, plates, sticks, or beads. Of course, other binding agents known to those of skill in the diagnostic assay art may also be employed for the same purposes. Other reagents include conventional diagnostic labels or label systems for direct or indirect labeling of the antibodies, peptides or nucleic acid sequences, with e.g., radioactive compounds, radioisotopes, such as ³²P, ¹²⁵I, tecnhicium; fluorescent or chemiluminescent compounds, such as GFP, FITC, rhodamine or luciferin; and proteins such as biotin or enzymes and enzyme co-factors, such as alkaline phosphatase, beta-galactosidase or horseradish peroxidase; and/or molecular labels such as FLAG, etc.

Still another method useful in diagnosing melanoma or determining its clinical prognosis in a mammalian subject comprises detecting or measuring a change in the amount or level of nucleic acid expression or activity or protein expression or activity of one or more of the biomarkers VEGF, M-CSFR, GPMNB, M-CSF, and CCL-2 in a biological sample of the subject and determining the status of disease relative to a control. In one embodiment, this method is performed when the subject is receiving anti-cancer therapy. A typical anti-melanoma therapy is a BRAF inhibitor. Still other anti-melanoma therapies are known to one of skill in the art and anticipated to be useful in this assay.

In one embodiment, a control or reference standard useful in the assay is the amount or level of VEGF, M-CSFR, GPMNB, M-CSF, or CCL-2 in a healthy subject or in the same subject at an earlier time in therapy. Use of this method to detect an increase in the amount or levels of one or more of these biomarkers during the course of therapeutic treatment indicates the need for a change in treatment.

Yet another embodiment of a diagnostic method is a method of diagnosing melanoma or determining progression or clinical prognosis of melanoma in a mammalian subject by measuring the amount or level of tumor-infiltrating macrophages (TAMs) or a biomarker of these macrophages or a product secreted from said macrophages in a biological sample of the subject and determining the status of the melanoma relative to a control. In one embodiment, this method is performed when the subject is receiving anti-cancer therapy. A typical anti-melanoma therapy is a BRAF inhibitor. Still other anti-melanoma therapies are known to one of skill in the art and anticipated to be useful in this assay. In this method, a useful control or reference standard is the amount or level of said macrophages, biomarker or secreted product in the same subject's biological material at a different timepoint. In one embodiment, the control uses a timepoint which is an earlier time in therapy. Still other embodiments include use of controls which are standards developed from a population of similar cancer patients undergoing similar therapies. According to this method, an increase in the amount or levels of one or more of the macrophages, biomarkers or secreted products during the course of therapeutic treatment indicates the need for a change in treatment.

Diagnostic reagents can be readily selected or designed to detect the growth factors, proteins, receptors identified above, and known to be present on macrophages.

Diagnostic kits containing reagents suitable for use in the above diagnostic methods can also contains miscellaneous reagents and apparatus for reading labels, e.g., certain substrates that interact with an enzymatic label to produce a color signal, etc., apparatus for taking blood samples, as well as appropriate vials and other diagnostic assay components.

### IV. METHODS FOR DETERMINING EFFICACY OF TREATMENT

In yet another aspect, a method is described for determining the efficacy of targeted cancer therapy. Such a method involves administering to a mammalian subject in need thereof a therapeutic treatment directed at inhibiting a targeted signaling pathway that enhances growth of a cancer or tumor cell. For example, certain therapeutics can target one of the pathways discussed herein, such as the MAPK pathway. The method then employs a suitable assay conducted on a biological sample of the subject to determine if that same targeted pathway is paradoxically activated in non-tumor cells of the subject. Activation of the targeted pathway is detected by measuring the expression or activity of a gene or protein in the pathway or produced by activation of the pathway. The selection of suitable assays is within the ability of one skilled in the art and will depend upon the particular therapy and particular pathway targeted by the therapeutic.

For example, if the MAPK pathway is targeted, the assay may measure a pathway activation indicator, e.g., the production or activation or activity of ERK, or an analogous protein involved in the MAPK pathway or produced thereby. When the assay detects that the targeted pathway has been activated in non-tumor cells of the subject, at any time during the course of the therapeutic treatment, this measurement or detection of the pathway indicator indicates a lack of efficacy of the current therapeutic treatment. This lack of efficacy can develop over time as the subject's body accommodates to the therapy or alternatively reflect a negative side effect of the therapeutic treatment. The result indicates the need for a change in therapy.

In one exemplary embodiment of this method, the cancer is melanoma and the therapeutic treatment is BRAF inhbitors. One suitable targeted pathway is the MAPK pathway; and the pathway gene or protein which is assayed in normal cells is ERK. However, one of skill in art can employ this method to determine the efficacy of targeted pathway therapeutics for other cancers and other diseases according to the teachings of this specification, including the examples.

### V. ASSAY TO DIFFERENTIATE MONOCYTES TO MACROPHAGES

Use of conditioned media from tumor cells are an established method to differentiate primary human monocytes to TAMs. However, there are limitations with the currently used methods. For example, Solinas et al. (2010) reported that culture media from only two of 16 tumor cell lines (after 1 day of culture) were able to differentiate monocytes to macrophages.

Thus in another aspect, the inventors provide an efficient in vitro assay to differentiate human monocytes to macrophages. This assay facilitates study of the functions of TAMS. An example of such an assay is illustrated in Example 1 below. The assay method involves culturing monocytes in concentrated melanoma tumor cell derived conditioned medium (MCM). The modified, concentrated MCM is produced by culturing melanoma cells, such as C8161 and 1205Lu melanoma cells, in melanoma media. The medium is supplemented with fetal bovine serum (FBS). In certain embodiments, the FBS is present in the medium at 1, 2, 3, 4, 5, 6, 7, 8, 9, or about 10% w/w. The melanoma cells are cultured for 2, 3, 4, or about 5, or up to about 7 days. MCM is harvested and concentrated between about 10 fold to about 100 fold. In one embodiment, the MCM is harvested at about 20-fold, 30-fold, 40-fold, 50-fold, 60-fold, 70-fold, 80-fold, 90-fold or about 100-fold. In one exemplary concentration the MCM is concentrated 40-fold. The MCM may be concentrated using a Centrifugal Filter Device from Millipore with a desired pore size, e.g., Example 6 uses a pore size of 10 kD. Other pore sizes may also be desirable.

The concentrated MCM is added to a complete medium, such as RPMI 1640 medium, optionally supplemented with FBS (see Example 6) at a suitable ratio to make the modified concentrated MCM. In certain embodiments, the FBS is present in the medium at 1, 2, 3, 4, 5, 6, 7, 8, 9, or about 10% w/w. A suitable ratio of concentrated MCM to complete medium may be between 1:50 and 1:200. Suitable ratios thus include 1:50, 1:60, 1:70, 1:80, 1:90; 1:100, 1:110, 1:120, 1:130, 1:140, 1:150, 1:160, 1:170, 1:180, 1:190; or up to about 1:200. In one embodiment a suitable ratio is 1:50. In another embodiment a suitable ratio is 1:80.

Thereafter monocytes are seeded in tissue-culture treated plates and are incubated in the presence of concentrated MCM derived from the cultured melanoma cells for about a week. In one embodiment, enriched monocytes are obtained from healthy donors, e.g., by leukapheresis followed by countercurrent elutriation. In one embodiment about 30, 40, 50, 60 70, 80 90 to 100% of media were changed in each plate on day three. Still other embodiments, involve changing the media on another day or on multiple days during incubation.

To generate M1-MΦ, M2-MΦ, and dendritic cells (DCs), monocytes are incubated for a suitable time, e.g., 2, 3, 4, 5, 6, 7, 8, 9 or up to 10 days, in the concentrated MCM in presence of selected factors. In one embodiment the suitable time is 7 days incubation. In one embodiment a selected factor is one or more of M-CSF, M-CSF, or M-CSF, and optionally IL-4.

Various of the conditions of this assay may be modified by one of skill in the art. As shown in the examples below, using 3 day concentrated MCM, the inventors are able to consistently differentiate monocytes. Under these conditions, more cytokines, including M-CSF, are produced compared with 1-day culture media (data not shown). Also, filtration of the concentrated culture media appears to retain the growth factors needed for TAMs differentiation, while filtering out cell culture metabolites that are acidic and other small molecular weight toxic metabolites that may affect monocyte/macrophage survival.

### VI. EXAMPLES

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

The phenotype and biological significance of macrophages in melanoma progression remains poorly characterized, especially in humans. In the following examples, the inventors demonstrate a novel method to consistently differentiate human monocytes to macrophages using melanoma-conditioned media. These macrophages share many characteristics with tumor-associated macrophages. Importantly, using these induced macrophages, it was determined that combinations of blocking both CCL2 and matrix metalloproteases inhibits macrophage-induced melanoma invasion. Additionally, glycoprotein non-metastatic melanoma protein B (GNMPB) was identified as a novel marker for TAMs. These findings provide new insights into the roles of TAMs in melanoma progression and metastasis and the potential for targeting TAMs as novel therapeutic strategies for melanoma progression.

To better characterize TAMs, we developed a highly efficient in vitro method to differentiate human monocytes to macrophages using modified melanoma-conditioned media (MCM). We demonstrate that factors from MCM-induced macrophages (MCMI-MΦ) express both M1-MΦ and M2-MΦ markers and inhibit melanoma-specific T-cell proliferation. Microarray analysis on these MCMI-MΦ showed that many genes associated with melanoma cell invasion and metastasis were up-regulated. The majority of genes up-regulated in MCMI-MΦ (Table 1) are associated with tumor invasion. The most strikingly up-regulated genes are CCL2 and MMP-9. MCMI-MΦ were able to increase melanoma cell invasion in vitro. Blockade of both CCL-2 and MMPs significantly inhibit or diminish MCMI-MΦ-induced melanoma invasion, even though there was no inhibitory effect by either factor alone. Finally, through microarray analysis and tissue staining, we demonstrate that TAMs (both MCMI-M4 and in vivo TAMs) present in human melanomas highly express the pro-invasive, melanoma-associated gene, glycoprotein non-metastatic melanoma protein B (GPMNB). This method enables one to understand the roles of TAMs in melanoma progression and metastasis and for understanding the mechanisms of cross-talk between TAMs and melanoma cells within the tumor microenvironment.

Macrophages play a role in promoting cancer development and are observed in melanomas. The inventors have determined that macrophages, a major component of tumor microenvironment, are activated by targeted therapies⁶⁻¹⁰ to promote resistance to these therapies. We have demonstrated that specific BRAF^{V600E} inhibitors (BRAFi) used in the treatment of mutant BRAF^{V600E} melanomas promote relapse through activation of tumor-associated macrophages. In the presence of BRAFi, macrophages promote tumor growth and survival by activating the mitogen-activated protein kinase (MAPK) pathway in melanoma cells through the production of vascular endothelial growth factor (VEGF). Blockade of VEGF signaling reverses macrophage-mediated resistance. BRAFi also strongly paradoxically activate the MAPK pathway in macrophages due to high basal level of RAS activation, leading to potent induction of VEGF production, thus creating positive feedback for both macrophages and melanoma cells. The importance of macrophages to resistance was further confirmed by using a human xenograft model treated with BRAFi and a macrophage-colony stimulating factor receptor inhibitor. The presence of abundant macrophages in melanomas prior to targeted therapies predicts macrophage-mediated resistance to targeted BRAFi.

### Example 1

### Methods and Materials for Examples 2-12

A. Differentiation of Human Monocytes to Macrophages

Enriched monocytes were obtained from healthy volunteers by leukapheresis followed by countercurrent elutriation (AIDS Research Human Immunology Core at the University of Pennsylvania). Monocyte purity was >94% as confirmed by FACS analysis (Becton Dickinson). To produce the modified MCM, C8161 and 1205Lu melanoma cells were seeded in 10-cm plates at 50% confluence and were then cultured in melanoma media supplemented with 2% FBS for 3 days. MCM was harvested and concentrated 40-fold using Centricon concentrators (Millipore). Concentrated media were added to complete RPMI 1640 medium (R10 medium, RPMI, 10% FBS, 10 mM HEPES, 100 μM 2-mercaptoethanol, 100 IU penicillin G, and 100 μg/ml streptomycin) at a 1:80 ratio to make the modified MCM.

For MCMI-MΦ differentiation, 2×10⁶ monocytes were seeded in tissue-culture treated 6-well plates (BD-Falcon) and were incubated in the presence of concentrated MCM derived from 1205Lu or C8161 melanoma cells for 7 days at 37° C. in a humidified atmosphere of 5% CO₂. About 50% of media were changed in each plate on day three. The supernatants were harvested for the detection of cytokines and chemokines. To generate M1-MΦ, M2-MΦ, and dendritic cells (DCs), monocytes were incubated for 7 days in the presence of M-CSF (10 ng/ml), M-CSF (10 ng/ml), or M-CSF plus IL-4 (10 ng/ml, R&D Systems) in R10 medium for 7 days, respectively. About 50% of media were changed in each plate on day three. For the M-CSF blocking experiment, monocytes were incubated in the presence of C8161 MCM and 1205Lu MCM with anti-human M-CSF (R&D Systems, 10 μg/ml) for 7 days.

B. Flow Cytometric Analysis

The following fluorescence conjugated antibodies were used for cell surface staining: anti-Ig mouse isotype control, anti-CD14, anti-CD68, anti-CD163, anti-CD 11b, anti-CD115, and anti-CD11d (all from Biolegend). Cells were acquired using a FACSCalibur™ apparatus and data were analyzed using FlowJo™ software.

C. Multiplex Cell Signaling Bead-Based Luminex Assays

The production of M-CSF, CCL2, IL-6, LIF, VEGF-A, and M-CSF from MCM, and of TNF-α, IL-12, IL-10, CCL1, CCL2, CXCL5, and CCL8 from 1205Lu-MCMI-MΦ (1205Lu-MΦ) and C8161-MCMI-MΦ (C8161-MΦ) was measured using the customized MILLIPLEX MAP Cytokine Kit according to the manufacturer's protocol (Millipore). Median fluorescence intensity was calculated from duplicates of each sample. Samples were analyzed using the Bio-Plex suspension array system (Bio-Rad Laboratories).

D. Microarray Data Generation and Analysis

Total RNAs were extracted using the TRizol reagent (Invitrogen) from monocytes (duplicate) and from C8161-MΦ (triplicate). cDNAs were generated, fragmented, biotinylated, and hybridized to the Illumina HumanHT-12V4 expression Beadchip Arrays (Illumina). The

detailed microarray data analysis procedure is noted in Supporting Information.

E. Inhibition of T-Cell Proliferation in Coculture Assays

The ability of MCMI-MΦ to inhibit anti-CD3 induced T-cell proliferation was determined using a coculture assay as described previously (Somasundaram et al., 2002). Briefly, inhibition of proliferation of antimelanoma reactive CTL793 and 35Th1 (5×10⁴) were

determined by coculturing T cells in the presence or absence of 1205Lu-MΦ at various ratios. The proliferation of T cells was determined using a standard 3H-TdR incorporation assay and the % inhibition of T-cell proliferation was determined as described earlier (Somasundaram et al., 2002).

F. Real-Time PCR (RT-PCR)

For RT-PCRs, 1 μg DNA-free RNA was used with oligo(dT) primers and Superscript reverse transcriptase. Transcripts of the house-keeping gene GAPDH in the same incubations were used for normalization. Oligonucleotides specific for CCL2, CCL8, CXCL5, CCL7, MMP7, MMP-9, GPMNB, and GAPDH are listed in Table 2. The primers were designed according to the Roche software for quantitative real-time PCR (Roche).

TABLE 2
REAL-TIME PRIMER SEQUENCES
FOR INVASIVE SIGNATURE GENES

| | Forward | SEQ | Reverse | SEQ |
|---|---|---|---|---|
| Gene | Primer | ID | primer | ID |
| Symbol | 5′-3′ | NO | 5′-3′ | NO |
| CCL2 | CAAGCAGAAGTG | 1 | TCTTCGGAGTTT | 7 |
| | GGTTCAGGAT | | GGGTTTGC | |
| MMP9 | CAAGCAGAAGTG | 2 | TTCAGGGCGAGG | 8 |
| | GGTTCAGGAT | | ACCATAGA | |
| MMP7 | GCTGGCTCATGC | 3 | TCCTCATCGAAG | 9 |
| | CTTTGC | | TGAGCATCTC | |
| CCL8 | ACCTCTCATGCT | 4 | CAGAAGCGCTGC | 10 |
| | GAAGCTCACA | | AGAAACCT | |
| CXCL5 | CAGACCACGCAA | 5 | GGGCCTATGGCG | 11 |
| | GGAGTTCA | | AACACTT | |
| GPNMB | GGCCTGAAAGCT | 6 | CCTCCGTGGGAA | 12 |
| | CCCTAATAGACT | | TGCT | |

G. Immunoblotting of MMP-9 and GPMNB

M2-MΦ, 1205Lu-MΦ, and C8161-MΦ were harvested and incubated in the presence of R10 medium for two additional days. Conditioned media were harvested and subjected to 10% SDS-PAGE electrophoresis. After the protein transfer, PDVF membranes were blocked and incubated with anti-MMP-9 or anti-GPMNB antibodies. The signals were visualized with enhanced chemiluminescence reagents (Amersham Biosciences).

H. Gelatin Zymography

The same samples mentioned earlier were subjected to electrophoresis with 10% Novex Zymogram Gels (Invitrogen). After renaturing and developing the gels according to the manufacturer's instructions, they were stained with Collide Blue Stain Reagent (Invitrogen).

I. Invasion Assay

The invasion assay was conducted with 24-well Transwell inserts (8 μm pore size; Corning). For the MCMI-MΦ-induced invasion assay, 4×10⁵ 1205Lu melanoma cells in 2% FBS RPMI1640 medium were added in the upper chamber precoated with 50 μl Matrigel (1:3 dilution, BD Biosciences). Media from 1205Lu-MΦ were added to the lower chamber, and R10 medium was used as a control. After overnight incubation, cells that had invaded

were fixed and stained with Diff-Quick staining kit (ThermoFisher). The stained cells in 4 randomly chosen fields were counted for each insert (200×). For the blocking assay, either anti-CCL2 (20 μg/ml, R&D Systems) or the MMPs inhibitor, GM6001 (10 μM, EMD Biosciences), or combined both were added to the Transwell.

J. Human Tumor Samples, Immunohistochemistry, and Immunofluorescence

Formalin-fixed, paraffin-embedded human melanoma tumors and human breast cancer tumors were obtained from the University of Pennsylvania and from Nanjing Medical University, under an approved Institutional Review Board protocol.

### Example 2

### Macrophages Confer BRAF Mutant Melanoma Cell Line Resistance to PLX4720

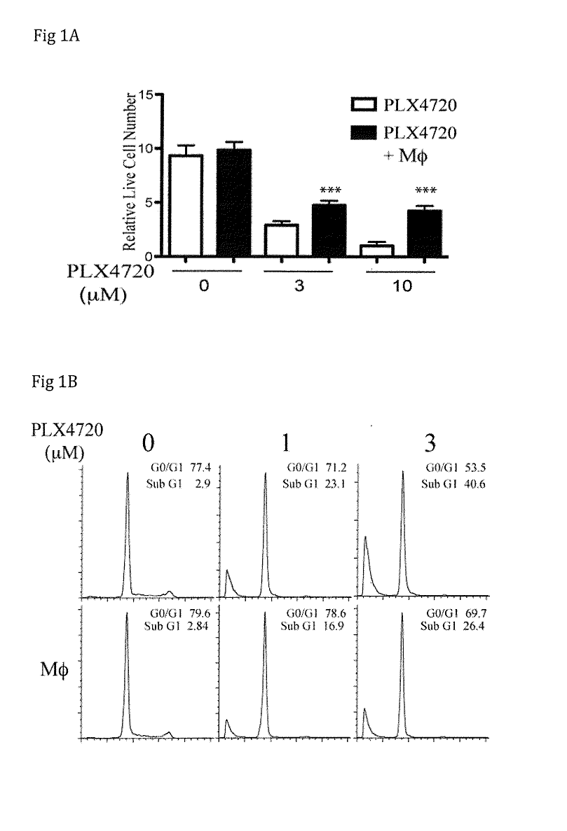

The metastatic melanoma cell line 1205Lu were seeded in the 24 well plate overnight. Cells were treated with the increasing concentrations of PLX4720 in the presence or absence of MCMI-MΦ for 72 hours using a transwell co-culture system. Melanoma cells were harvested and stained with trypan blue. Live cells were counted and the proportion of viable cells relative to the control was determined. The results are shown in the plot of FIG. 1A of Mean±SD (n 3) of the percent of viable cells versus the DMSO control. ***, P<0.01.

Cells were stained with propidium iodide and analyzed by flow cytometry. The proportion of cells with sub-G1 DNA content is indicated in FIG. 1B.

Cells were treated as in the first paragraph above for 2 days. Cell lysates were analyzed on Western blot (gel not shown). For the 8 columns of gel blots, the amounts of PLX4720 (0, 1, 3, and 10 μM) with both presence or absence of MΦ were indicated. In the absence of PLX4720, with or without macrophages, only a single blot appeared in the cPARP gel, whereas all other conditions showed a second blot.

### Example 3

### MCMI-MΦ Activate MAPK and PI3K/mTOR Pathways in PLX4720 Treated Melanoma Cells

1205Lu cells were treated with the BRAF inhibitor, PLX4720, in the presence or absence MCMI-Mφ for 6 and 18 hours. Cell lysates were analyzed by Western blot for phospho-ERK, total ERK, pRSK90, pAKT, pS6, pRSK90, phospho-4EBP1, pCRAF, pNF-KB P65. RAb11 or HSP90 was used as a loading control. The amounts of PLX4720 (0, 1, 3, and 10 μM with both presence or absence of Mφ were indicated in the 6 hour gel; only 1 and 10 μM were indicated with the 24 hour gel. The resulting gels (data not shown) demonstrate the 6 hour and 18 hour experimental periods and demonstrate activation of the MAPK and PI3K pathways, as shown by decreased size and signal of blots for pERK (T202/204), pAKT, and pS6 in both time points.

### Example 4

### Activation of MAPK and PI3K/mTOR Pathways Via MCMI-Mφ is Dependent on Activation of MCSF-R Signaling

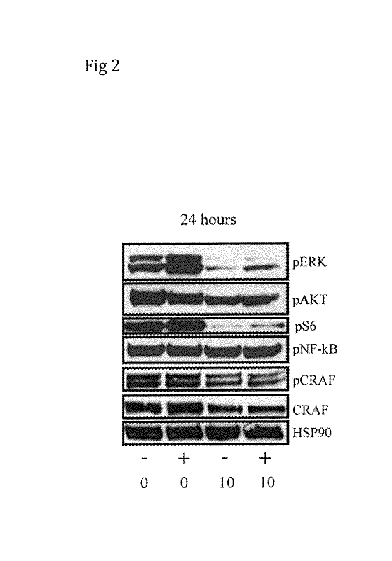

Cell lysates from melanocytes and melanoma cells (Fom 133, WM3211, WM35, WM98, WM793, WM9, WM75, 1205Lu and A375) were analyzed by Western Blot for M-CSFR expression. Rab11 was used as a loading control. The results are shown in the gel of FIG. 2A.

1205Lu were treated with the BRAF inhibitor (PLX4720; 10 μM) and the M-CSFR inhibitor (GW2580; LC Laboratories, 10 μM), alone or in combination, and soluble M-CSFR alone or combination with MCMI-Mφ for 3 days. Western Blot was performed to determine expression of pERK, pAKT, pS6, pRSK90. Rab11 was used as a loading control. The gel (data not shown) showed the expression of pERK (L) and (S), pAKT, pS6, pRSK90, using Rab11 as a loading control. In the absence of any treatment (col. 1) or in the presence of MCMI-Mφ only (col. 2), blots for all markers are displayed. In the presence of PLX4720 only (col. 3), there are no visible blots for pRSK90, pERK(S) or (L) and pS6. In the presence of MCMI-Mφ and PLX4720 (col. 4), the missing blots appear, but lighter than in cols. 1 and 2. In the presence of MCMI-Mφ and GW2580 (col. 5), all blots appear similar to col. 2. In the presence of GW2580 only (col. 6), all blots appear similar to col. 4. In the presence of PLX4720 and GW2580 in combination, (col 7), no visible blots are evident for pRSK90, pERK(s) and (L), and pS6. In the presence of MCMI-Mφ, PLX4720 and GW2580 (col. 8), col. 8 appears to be the same as col. 7. In the presence of m-CSFR only (col 9), no change from control is observed. In the presence of MCMI-Mφ and m-CSFR (col. 10), no change from col 9 is observed. In the presence of PLX4720 and m-CSFR (col. 11) or GW2580 and m-CSFR (col. 12), no visible blots are evident for pRSK90, pERK(s) and (L), and pS6.

Melanoma cells and macrophages were treated with PLX4720, M-CSFR inhibitor (GW2580) alone or combination, soluble M-CSFR alone or combination with MCMI-Mφ for 3 days. Cells were harvested, and PI staining was performed for flow cytometry analysis. Rab11 was used as a loading control.

### Example 5

### Gw2580 Increases Inhibitory Effect of PLX4720 on Melanoma Tumor Growth In Vivo

5×10⁵ 1205Lu cells were subcutaneously injected to Nude mice (Female, 8 weeks old). After tumors reached to 100 mm³, mice were dosed twice a day with PLX4720 (25 mg/ml), GW2580 (160 mg/ml) daily and a combination of PLX4720 and GW2580.

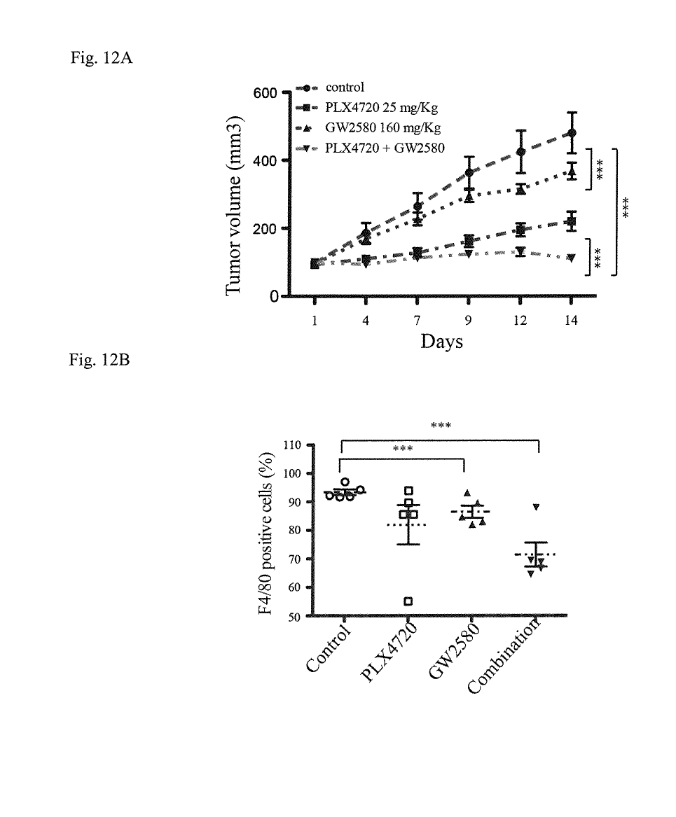

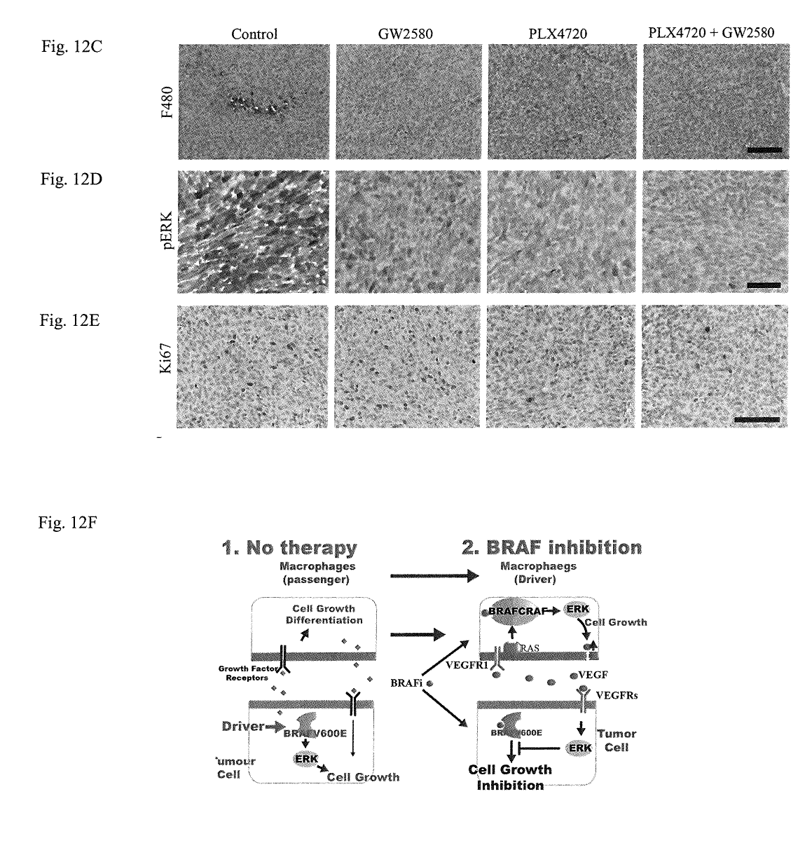

Tumor size was measured by a caliper. Tumor volume was calculated by the formula: Volume width×wide×length/2. The results are shown in the plot of FIG. 12A, plotting tumor volume vs. day.

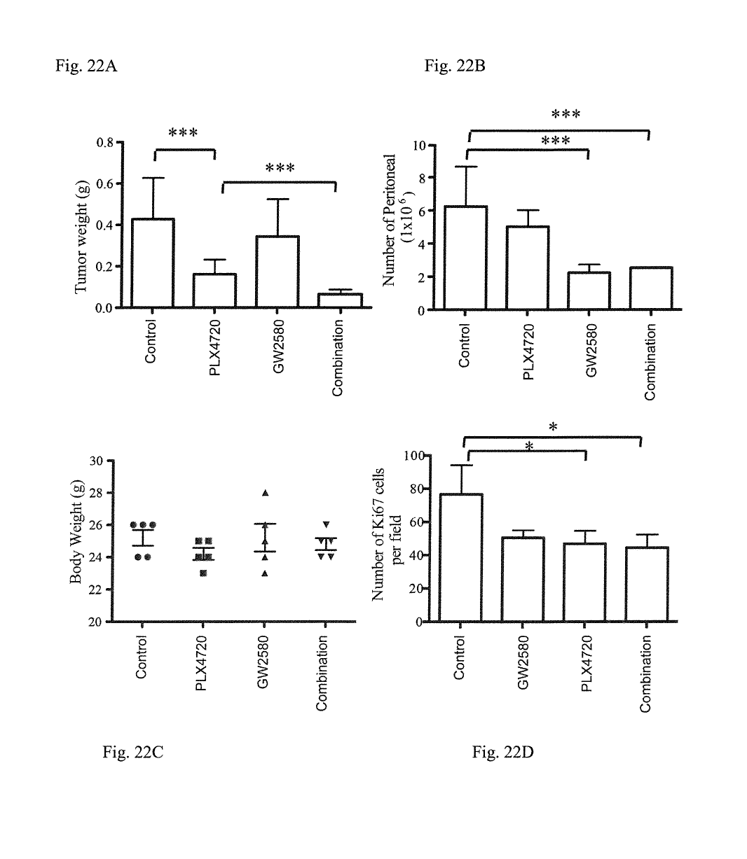

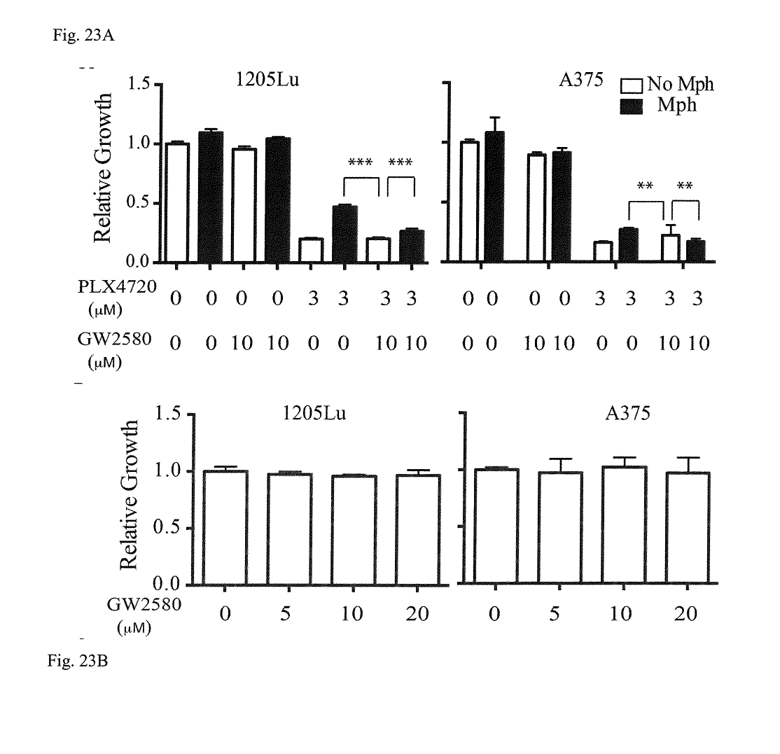

Tumors from the experiment described immediately above were extracted and weighted after mice were euthanized. Tumor weight in grams was plotted vs. control or an agent defined along the x axis of FIG. 22A.

Peritoneal cells were counted and plotted against the control or agents defined along the x axis of FIG. 22B.

Other peritoneal cells were harvested and stained with F4/80 for flow cytometry analysis. F4/80 positive cells are shown in the graph of FIG. 12B for each treatment agent along the x axis.

### Example 6

### Differentiation of Human MCMI-MΦ In Vitro with MCM

To differentiate monocytes to MCMI-MΦ, we concentrated 3-day MCM with a Centrifugal Filter Device from Millipore (pore size, 10 kD) and added the MCM to RPMI medium supplemented with 10% fetal bovine serum (FBS) at a 50% ratio of the original MCM. After 7 days of incubation, monocytes differentiated to MCMI-MΦ, based on cell morphology and the pattern of expression of macrophages/TAM markers. This effect was elicited by MCM from two non-metastatic melanoma lines, WM35 and WM793 (data not shown), as well as from two metastatic melanoma cell lines, 1205Lu and C8161.

We tested whether TCM from one ovarian cancer line, Ovca42, and two breast cancer cell lines, T47D and MD-MB-231, also differentiated monocytes to MCMI-MΦ. Similar to the MCM, TCM from these other cell lines also differentiated monocytes to macrophages (data not shown). These data indicate that this is a reliable method to differentiate monocytes to MCMI-MΦ.

### Example 7

### Characterization of MCMI-MΦ in Melanomas

We characterized the MCMI-MΦ by analyzing their morphology, expression of surface markers, cytokine/chemokine profile and function. Monocytes from healthy donors were cultured in the presence of GM-CSF (10 ng/ml), M-CSF (10 ng/ml), 1205Lu-MCM, or C8161-MCM for 7 days and differentiated to modified melanoma conditioned medium-induced macrophages (MCMI-MΦ, i.e., M1-MΦ, M2-M4, 1205Lu-MΦ, and C8161-MΦ. After 7 days of incubation, MCMI-MΦ that were differentiated by C8161 or 1205Lu MCM (C8161-1\44) and 1205Lu-M4)) showed in micrographs (data not shown) elongated shapes and typical spindle-like macrophage morphology, which is similar to the M2-MΦ, whereas M1-MΦ typically show a round, fried-egg shape as previously described (Svensson et al., 2011; Waldo et al., 2008).

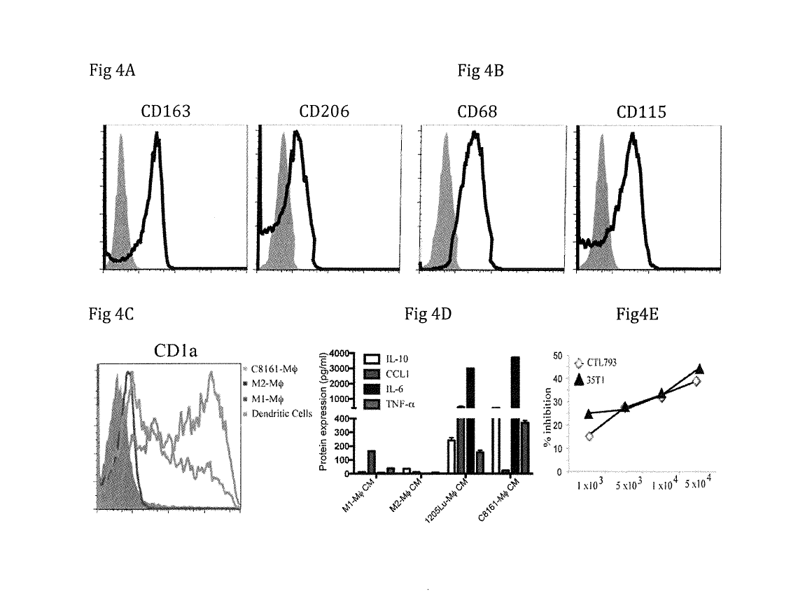

We then characterized the expression of macrophage surface markers by flow cytometry analysis. C8161-MΦ expressed the M2-MΦ markers, CD163 and CD206 (FIG. 4A). In addition, CD68 and CD115, which are expressed by both M1 and M2 macrophages, are also expressed by C8161-MΦ (FIG. 4B). Furthermore, C8161-M4 also expressed other M2-MΦ markers, such as CXCR4, CD16, and CD36 (data not shown). Neither C8161-MΦ nor 1205Lu-MΦ expressed the dendritic cell marker CD1a (FIG. CD and data not shown), indicating that C8161-MΦ and 1205Lu-MΦ are macrophage and not dendritic cell lineage.

To further characterize MCMI-MΦ based on factors they produce, we analyzed the production of cytokines and chemokines previously implicated to be expressed in M1-MΦ and M2-MΦ (Ilkovitch and Lopez, 2008; Payne and Cornelius, 2002). Both 1205Lu-MΦ and C8161-MΦ secreted high levels of the M2-MΦ cytokines, IL-10 and CCL1, as well as the M1-MΦ cytokines, IL-6, and TNF-α. No production of IL-12 (p40) was detected in 1205Lu-MΦ or C8161-MΦ (FIG. 4D and data not shown). Of note, 1205Lu-MΦ and C8161-MΦ produced more cytokines/chemokines than M2-MΦ, and there are significant differences in cytokines/chemokines produced by 1205Lu-MΦ and C8161-MΦ, further suggesting that MCMI-MΦ may be heterogenous and bear both M1 and M2 phenotypes. One of the major activities of TAMs is their ability to suppress antitumor immunity.

For example, it has been shown that macrophages were able to inhibit T-cell proliferation owing to expression of indoleamine 2,3-dioxygenase (Munn et al., 1999). To determine the potential ability of MCMI-MΦ to inhibit T-cell proliferation, we cocultured 1205Lu-MΦ with two different anti-melanoma reactive T-cell clones: a CD4 T-cell clone, 35Th1, and a CD8 T-cell clone, CTL793, each established as described earlier from peripheral blood lymphocytes of patients with melanoma. C8161-MΦ significantly inhibited T-cell proliferation to anti-CD3 stimulation both in CD4 and in CD8 T-cell clones in a dose-dependent manner (FIG. 4E). These data suggest that MCMI-MΦ are able to inhibit T-cell proliferation.

### Example 8

### Differentiation of MCMI-MΦ in Melanomas is not Dependent on M-CSF

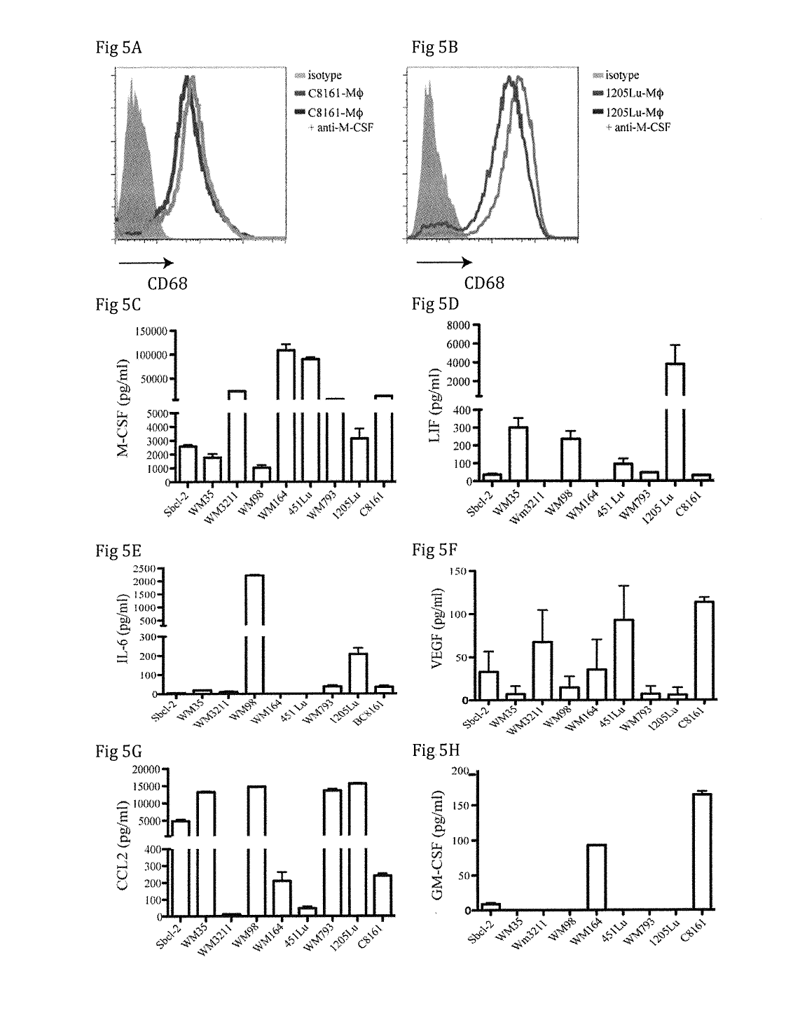

The differentiation of monocytes to PCMI-MΦ has been reported to be dependent on M-CSF (Solinas et al., 2010). To investigate whether MCMI-MΦ differentiation is also dependent on M-CSF, we incubated monocytes in the presence of C8161 MCM or 1205Lu MCM in the presence of anti-human M-CSF (10 μg/ml) or an isotype control antibody for 7 days. We observed a slightly decreased expression of CD68 in both C8161-MΦ and in 1205Lu-MΦ (FIGS. 5A and 5B). These data indicate that the differentiation of MCMI-MΦ is not only dependent on M-CSF, but that other factors may also play roles in MCMI-MΦ differentiation.

Melanoma cells produce factors in addition to M-CSF that are related to macrophage differentiation, including CCL2, M-CSF, VEGF-A, LIF, and IL-6. It is possible that

melanomas at different stages of development may produce very different TAMs based on their unique cytokine patterns. Therefore, we characterized cytokine/chemokine production in melanoma cell lines derived from melanomas at different stages: three radial growth phase melanoma lines (RGP): Sbc1-2, WM35, and WM3211, three vertical growth phase melanoma
lines (VGP): WM98, WM793, WM164, and three metastatic melanoma cell lines, 1205Lu, 451Lu, and C8161.

All melanoma cell lines produced M-CSF, CCL2, and VEGF-A, but at different levels. Seven of the nine cell lines produced LIF and IL-6, and only three of the nine cell lines produced GM-CSF, which is a major M1/differentiation factor. Of note, there was no pattern of cytokine production specific for different stages of melanomas (FIG. 5C through 5H), and therefore, the production of the different types of macrophage is not likely correlated with melanoma progression.

### Example 9

### Gene Profiling of MCMI-MΦ in Melanomas

To further characterize novel factors expressed in MCMI-MΦ, we performed microarray analyses to characterize the molecular gene signature of C8161-MΦ by comparing them with the normal monocyte gene profile. A total of 1912 genes were differentially regulated (1019 up-regulated and 893 down-regulated) in a total of 47 000 probes in C8161-MΦ (data not shown, see Data S1 and S2 of the Wang et al 2012 online publication).

Next, we compared the gene expression profiles using microarray data that are publically available in the GEO database (GSE). We found that 16.4 and 16% of up-regulated genes in C8161-MΦ overlapped with genes expressed in GSE for M1-MΦ and M2-MΦ, respectively, while 17.9 and 7% of down-regulated genes overlapped between M1-MΦ and M2-MΦ, respectively (data not shown; see Figs S2A and S2B of the Wang et al 2012 online publication). Therefore, MCMI-MΦ have a gene expression profile that is not characteristic of either M1-MΦ or M2-MΦ.

### Example 10

### An Invasive Signature in Melanoma MCMI-MΦ

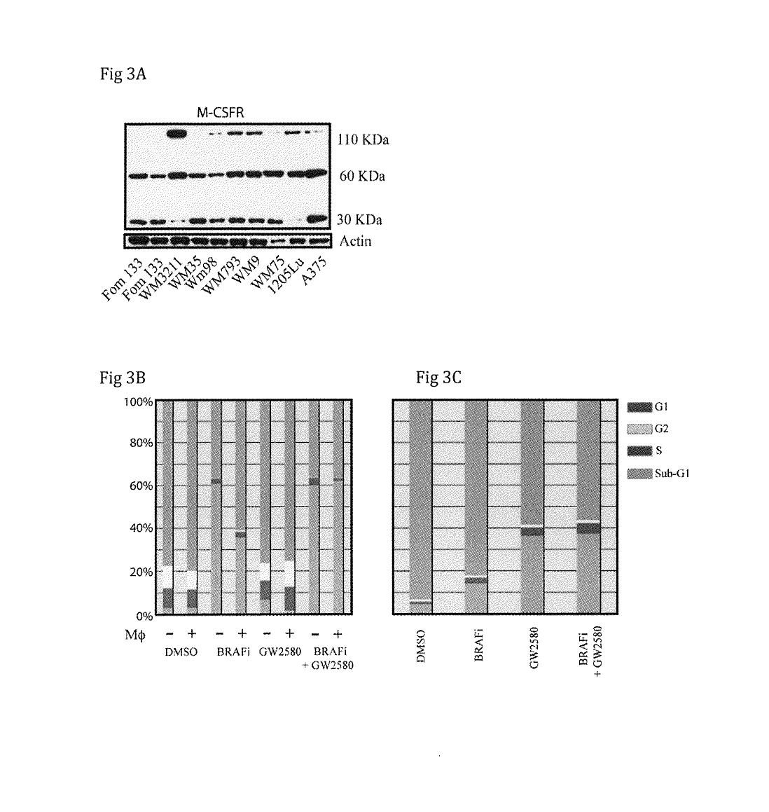

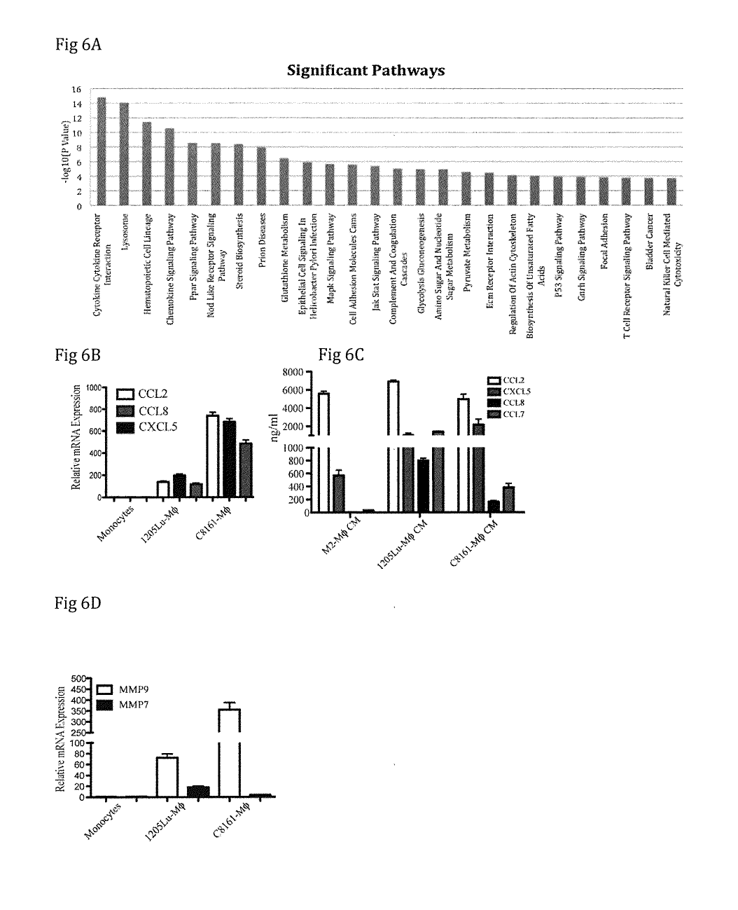

We performed pathway analysis of significantly up-regulated genes that were revealed by the gene expression profiling. Twenty-six pathways were found to be significant under a family-wise error rate (FWER) level of 0.05 (FIG. 6A). Among those pathways, seven are linked to cell metabolism, such as glutathione metabolism. Strikingly, nearly all other pathways have been implicated to play roles in tumor invasion and metastasis, such as cytokine/cytokine receptor interactions, chemokine-signaling pathways, cell adhesion molecules, the Jak-/Stat-signaling pathway, ECM receptor interactions, regulation of the actin cytoskeleton and focal adhesion molecules (data not shown; see FIG. 3(E) of reference 11).

A detailed analysis of the top 100 up-regulated genes revealed that most have been implicated in the promotion of tumor progression and metastasis, including 13 genes encoding chemokines and chemokine receptors (Table 1). A total of 20 chemokines are up-regulated in MCMI-MΦ (CCL2, CXCL5, CCL8, CCL7, CCL22, CCL42, CCL4L1, CCL20, CCL3, CCL13, CCL18, CCL3L1, CCL1, CCL23, CCL24, CXCL1, CXCL2, CXCL6, CXCL8 and CXCL16; see FIG. 3(E) of reference 11), and of note, CCL2 is the highest up-regulated gene. Real-time PCR analysis confirmed that the expression of CCL2, CXCL5, and CCL8 is up-regulated in C8161-MΦ (FIG. 6B). Furthermore, these chemokines were found to be more highly expressed in both C8161-M4 and 1205Lu-MΦ (FIG. 6C). Other up-regulated genes in MCMI-MΦ encoded proteases, including MMP-9, 7, 1, 12, and 14 secreted phosphoprotein 1 (SPP1, osteopontin), cathepsin L1 (CTSL1), and urokinase (uPA) (see FIG. 3(E) of Reference 11). In addition, a less studied molecule, GPMNB, which promotes tumor metastasis, is strongly up-regulated in MCMI-MΦ compared with monocytes. The up-regulation of MMP-9 and MMP-7 mRNA expression was verified by real-time PCR (FIG. 6D).

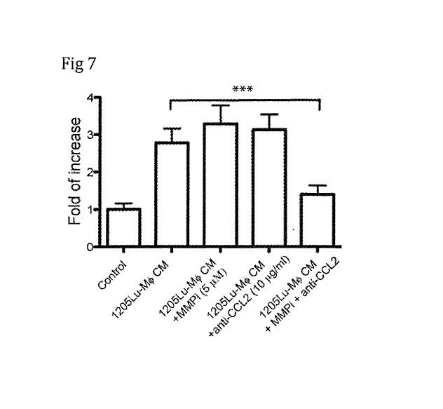

To confirm that MMP-9 was produced by MCMI-MΦ rather than the melanoma cell lines, we incubated 1205Lu-MΦ and C8161-MΦ with fresh 10% FBS RPMI1640 medium for an additional 2 days, and this macrophage-conditioned media supernatant was harvested for Western blot analysis. As shown in FIG. 7G (lower panel), 1205Lu-MΦ and C8161-MΦ produced high level of MMP-9. In addition, we found the activated form of MMP-9 in supernatants from 1205Lu-MΦ and C8161-MΦ by gelatin zymography (see FIG. 3(G) of Reference 11). In summary, these data indicate that MCMI-MΦ show an invasive signature.

### Example 11

### Blockade of Both MMPs and CCL2 Significantly Inhibit 1205Lu-MΦ-Induced Melanoma Invasion

As CCL2, MMP9, and MMP-7 are among the most up-regulated factors in the supernatants of MCMI-MΦ and are critical for melanoma invasion, we investigated the role of CCL2 and MMPs in the supernatants of 1205Lu-MΦ on melanoma invasion. We used a Matrigel Transwell assay and compared invasion of melanoma cells attracted to the supernatant of 1205Lu-MΦ alone or the supernatant with an anti-CCL2 monoclonal antibody and a pan-MMPs inhibitor GM6001 (see FIG. 7; see also FIG. 4(A)-(E) of Reference 11). The supernatant alone significantly increased melanoma cell invasion compared with control media. Surprisingly, instead of inhibiting 1205Lu-MΦ-induced invasion, treatment with either anti-CCL2 (10 μg/ml) or GM6001 (10 μM) did not have a significant effect on 1205Lu-MΦ supernatant induced melanoma invasion, while combined treatment with both significantly inhibited 1205Lu-MCM-induced invasion. These data indicate that the combination of the anti-CCL2 antibody and the MMPs inhibitor can achieve significant inhibition of MCMI-MΦ-induced melanoma invasion in Matrigel.

### Example 12

### MCMI-MΦ have an Invasive Signature Similar to TAMs

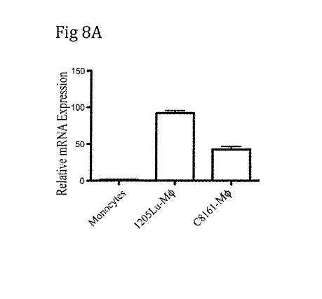

Expression of chemokines and proteinases in melanomas and macrophages has been well documented. These experiments have revealed a potentially important additional gene, GPMNB, which has not been reported to be expressed in TAMs. GPMNB is one of the top five ranked up-regulated genes in MCMI-MΦ (Table 1). Real-time PCR analysis revealed an 80-fold and 49-fold increased expression of GPMNB in 1205Lu-MΦ and in C8161-MΦ compared with monocytes, respectively (FIG. 8). Western blot analysis confirmed that GPMNB is expressed in M1-MΦ and in M2-MΦ, as well as in 1205Lu-MΦ and in C8161-MΦ (see, FIG. 5(B) in Reference 11).

To further characterize the expression of GPMNB in TAMs from melanoma lesions, we performed immunohistochemical staining with an anti-GPMNB antibody. Most GPMNB-positive cells were inflammatory cells (see, FIG. 5(C) in Reference 11), and few were melanoma cells (data not shown). We further performed double staining of GPMNB with the most commonly used TAMs markers, CD68 and CD163, to confirm whether GPMNB is expressed in TAMs. GPMNB was expressed in most CD68-positive cells, but there was not a complete overlap with CD68 staining (see, FIG. 5(D) in Reference 11). Presumably, some melanoma cells were also CD68-positive, as reported previously (31). As expected, most GPMNB-positive cells were CD163-positive ((see, FIG. 5(E) in Reference 11)). These data identify GPMNB as a novel marker for TAMs.

As GPMNB has been implicated in the promotion of breast cancer metastasis, we evaluated whether GPMNB is expressed in TAMs in breast cancer tissues. Immunohistochemistry staining confirmed that GPMNB is expressed in breast cancer lesions with most GPMNB-positive cells having a macrophage morphology (see, FIG. 5(F) in Reference 11)), and few cancer cells stained positive (data not shown). Furthermore, most GPMNB-positive cells were CD68 and CD163 positive (see, FIG. 5(G) in Reference 11)). Collectively, the in vivo expression of GPMNB further supports the invasive signature we have developed for MCMI-MΦ.

### Summary of Data in Example 1-12:

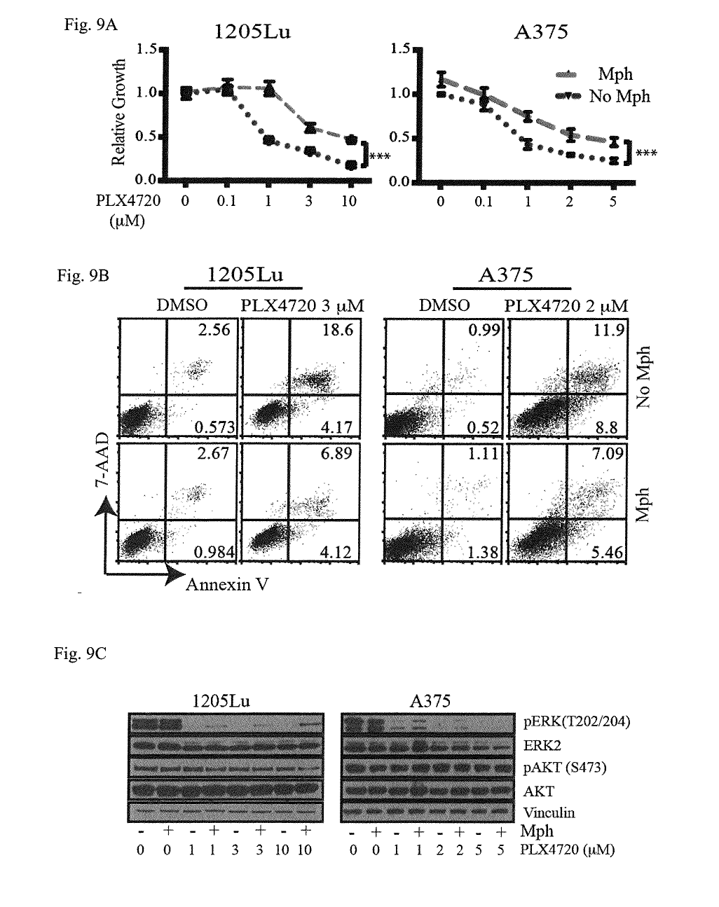

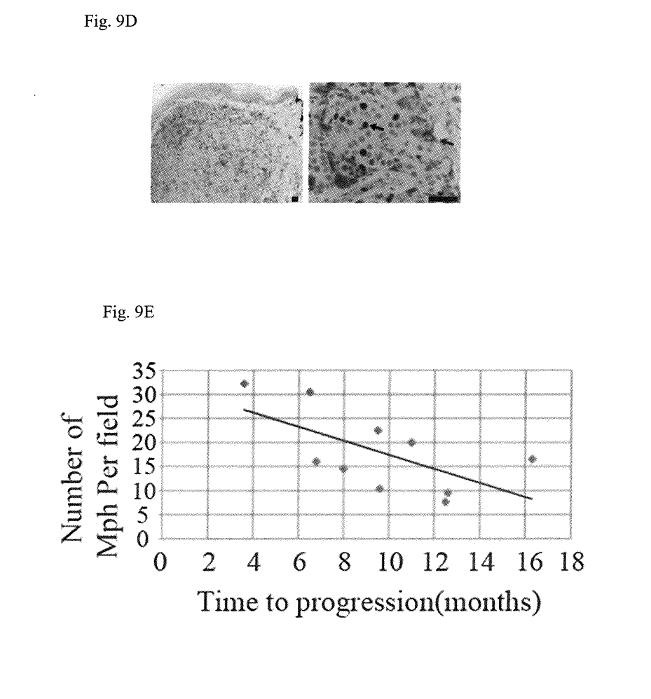

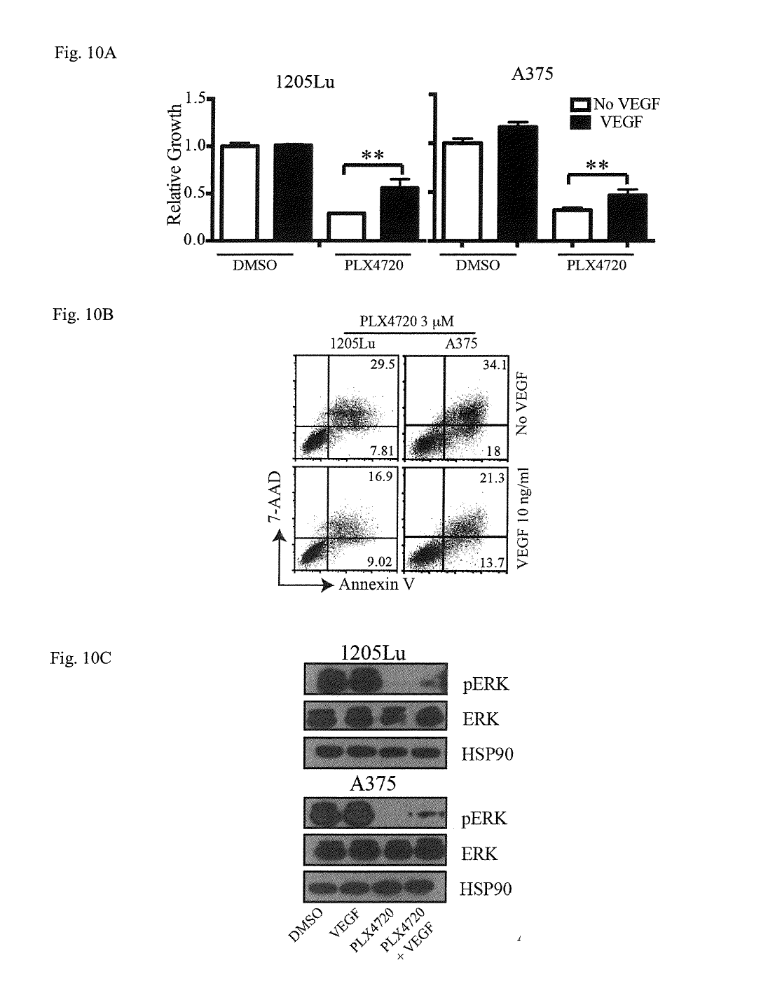

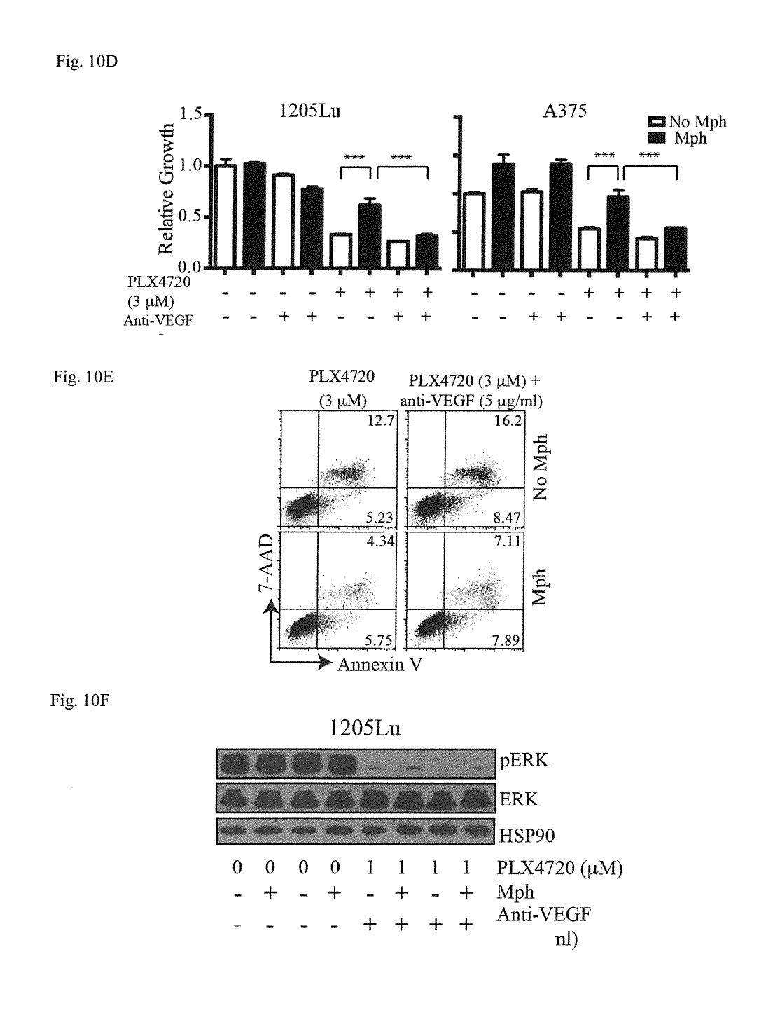

MCMI-MΦ produced by the assay methods described herein are similar to TAMs found in cancer tissues by gene profiling in vitro and in vivo (FIGS. 7 and 9) and by functional studies (FIGS. 5F and 8). In addition to having an invasive phenotype, we have identified several genes expressed in both types of macrophages that may be important in TAM function. In particular, several MCMI-MΦ up-regulated genes were identified, including DFNA5, that have not been previously reported to be expressed in the monocyte/macrophage lineage (FIG. 10), and which were also found in melanoma tissue TAMs (data not shown). Finally, we demonstrated that 3-day conditioned media from breast and ovarian cancer cells can also successfully differentiate monocytes to macrophages (data not shown).

The prevailing M1-MΦ and M2-MΦ differentiation model probably does not fully reflect the complexity of macrophages in the tumor microenvironment. Recent studies have also demonstrated that TAMs are a heterogeneous population and share both phenotypes of M1-MΦ and M2-MΦ (Mosser and Edwards, 2008; Umemura et al., 2008). In our model, we also found that TAMs in melanomas expressed both M1 and M2 markers and secrete multiple cytokines and chemokines associated with both M1-MΦ and M2-MΦ. MCMI-MΦ also produced M1-MΦ cytokines/chemokines, such as IL-1α, IL-6, and TNF-α (FIGS. 5E and 10). Our functional studies demonstrated that MCMI-MΦ are immunosuppressive and promote melanoma invasion, a definition of M2-MΦ. Supporting this, GPMNB, a pro-invasion gene, is expressed in M1-MΦ, M2-MΦ, and MCMI-MΦ. Collectively, our data support the concept of melanoma TAMs heterogeneity with both M1 and M2 phenotypes (Biswas et al., 2008).

Previous studies have shown that melanoma cells express factors related to TAM differentiation, but it is not known if the phenotype of TAMs is different in melanomas of different stages. Here, we show that there is no significant difference in the production of M-CSF, LIF, IL-6, VEGF, CCL2, or GM-CSF between cell lines from different stages of melanomas, consistent with the action of TAMs, which appear to be involved in every step of melanoma progression. Furthermore, multiple factors appear to be involved in TAMs development, consistent with our data that neutralization of M-CSF alone has a minimal effect on TAMs differentiation in melanomas (FIG. 6B). MCM from a panel of melanoma cell lines representing different stages of melanoma progression were able to differentiate monocytes similarly. While most melanoma cell lines do not express GM-CSF, some cell lines express both GM-CSF and M-CSF (FIG. 6B), perhaps partially explaining the heterogeneity of the phenotype in MCMI-MΦ.

Our findings suggest that there is heterogeneity in MCMI-MΦ, and the differences in secreted products of tumors contribute to this heterogeneity. Despite this, we found many gene pathways that are associated with an invasive phenotype to be up-regulated in MCMI-MΦ, especially those involving chemokines and MMPs. We also found that dual blockade of MMPs and CCL2 is required to block melanoma cell invasion promoted by MCMI-MΦ. These data may help to explain why inhibition of MMPs alone has little efficacy on the clinical outcome of patients with cancer in several clinical trials. A possible explanation is that MMPs and CCL2 have a positive feedback effect on each other. Blocking one of them might not be sufficient to block this positive feedback. MMPs and CCL2 are two major drivers for TAMs-induced melanoma invasion and provide a rationale to targeting both for melanoma therapy.

Previous work by Solinas et al. (2009) indicated that PCMI-MΦ also has an invasive signature. For example, SEPP1, osteoactivin, and GPMNB are among highest up-regulated genes in PCMI-MΦ, which are also significantly up-regulated in MCMI-MΦ. We see several differences in the phenotype of macrophages produced from PCMI compared with MCM. For example, there is a 240-fold increase in MMP-9 expression in MCMI-MΦ, which was increased ninefold in that other study. Other genes, up-regulated in PCMI-MΦ such as MMP-2, were not identified in our array list. Furthermore, unlike MCMI-MΦ, fewer chemokines and cytokines were up-regulated in that report. This may be because the conditioned media are different between cancer types or because of differences in the methods used to produce the different conditioned media. The efficacy in macrophage induction with our conditioned media from different tumor types suggests that this may explain the differences noted in the two studies.

There is no report of GPMNB expression in melanoma lesions in vivo. Based on the high level of up-regulated expression of GPMNB in our microarray analysis, we stained melanoma tissues for GPMNB and observed that the majority of GPMNB-positive cells are macrophages, and only a few melanoma cells express GPMNB. Similar results were also observed in breast cancer tissues (FIGS. 9C, 9F). This is consistent with the report (Rose et al., 2010) that 70% of GPMNB-positive cells are located in the stroma, and only 10% of GPMNB-positive cells are breast cancer cells.

The inhibitory effect of treatment with an anti-GPMNB antibody on melanoma tumor growth in vivo may be explained by the fact that it targets both TAMs in the tumor stroma and tumor cells. Furthermore, Solinas et al. (2010) also found in their microarray analysis that GPMNB is expressed in PCMI-MΦ (Solinas et al., 2010). Our data support the invasive signature of TAMs induced from MCMI-MΦ.

### Example 13

### Methods and Materials for Examples 14-19

Melanoma Co-Culture System

In summary, the macrophages and melanoma cell co-culture system is designed as follows. Melanoma cells were seeded onto the bottom of cell culture plates. To mimic the tumor microenvironment, a layer of collagen I was coated on the transwell. Macrophages were then seeded onto the collagen I. Macrophages are differentiated with melanoma-conditioned medium and display similar gene signatures and functions to tumor-infiltrating macrophages. This system allows the interaction between melanoma cells and macrophages through soluble factors.

Specifically, 1×10⁵ melanoma cells were seeded in the six well plate and incubated for 18 hours. 2×10⁵ macrophages were then added to the collagen I coated transwell (pore size: 0.4 μm) and culture for additional two hours. Indicated concentrations of various inhibitors, growth factors and antibodies were added to the co-culture system and incubated for indicated times. Melanoma cells and macrophages were harvested for Western blot analysis after six hours incubation, and for proliferation and cell death assay after 72 hours incubation.

Cell Culture

1205Lu, A375, SK-MEL-28 and 451Lu melanoma cells were cultured in melanoma medium supplemented with 2% fetal bovine serum (FBS) as described previously. Melanoma conditioned medium derived macrophages were produced as described previously.

Reagents

PLX4720, Lenbatiniab, Brivanib Alaninate were from Selleck. Dabrafenib was from Chemblink. Trametinib was from ChemieTek, GW2580 was from LC Laboratories. VEGF, anti-VEGF blocking mAb and phosphor-VEGFR1 (Y1213) were from R&D Systems. Corning Transwell was from Fisher Scientific for co-culture experiments.

Proliferation Assay

Melanoma co-culture system was set up as described above. For macrophage proliferation, after monocytes were differentiated into the macrophages, cells in 2% FBS melanoma media were seeded into 96 well plate and incubated for 3 days in the presence of indicated concentrations of inhibitors and blocking antibodies. Cell proliferation was assayed using the WST-1 proliferation kit (Roche) according manufacturer's instruction. All experiments were performed in at least triplicate.

Immmunoblotting

Macrophages and melanoma co-culture system was set up as described above. Melanoma cells were cultured same as Proliferation assay for 6 hours and were harvested for Immmunoblotting with following antibodies: phspho-ERK, total ERK, HSP90, phosphor-AKT, AKT, phosphor-NF-κB, phospho-CRAF, total CRAF, Phospho-ARAF, RAB11, Vinculin.

For macrophages, after monocytes were differentiated to the macrophages, cells in 2% FBS melanoma media were seeded into 15 ml tubes. Macrophages were incubated for indicated times in the presence of indicated concentrations of PLX4720 or MEK inhibitors. Immunoblotting were performed as described previously. The following antibodies were used: anti-phospho-ERK, ERK, Phospho-VEGFR1, phospho-CRAF, CRAF, PCNA, HSP90 and Rab11.

Flow Cytometric Analysis

For cell death assay, melanoma cells or macrophages were stained with R-Phycoerythrin conjugated Annexin V and 7-AAD, and evaluated for apoptosis and necrosis by flow cytometry according to the manufacturer's protocol (BD Biosciences). The apoptotic cells were quantified using a Becton Dickinson FACScan cytometer. Both apoptotic (annexin V-positive and 7-AAD-negative) and necrotic apoptotic (annexin V-positive and 7-AAD-positive) cells were included in cell death determinations.

For cell cycle analysis, melanoma cells were co-cultured with macrophages as described above, cells were fixed in 75% ethanol at −20° C. overnight. Cells were washed with cold PBS, treated with 100 μg of RNase A (Sigma), and stained with 50 μg of propidium iodide (Roche).

Measurement of VEGF production was determined by intracellular staining according to the manufacturer's protocol (BD Biosciences). After monocytes were differentiated to the macrophages, cells in 2% FBS melanoma media were incubated for 4 hours in the presence of the indicated concentration of PLX4720 or MEK inhibitors and GolgiPlug. After cells were washed with FACS buffer, intracellular staining was performed with R-Phycoerythrin conjugated anti-VEGF mAbs according to the manufacturer's instruction (R&D Systems). All flow cytometric data were analyzed with FlowJo software (TreeStar).

RAS Activation

After macrophages differentiation, cells in 2% FBS melanoma media were incubated for additional 1 hour. Cells were harvested for ELISA assay according to manufacturer's instruction (Millipore).

Patient Samples

Formalin-fixed, paraffin-embedded human melanoma tumor tissue slides were from the University of Pennsylvania under an approved Institutional Review Board protocol. The study was conducted in compliance with regulations of the Health Insurance Portability and Accountability Act and the Declaration of Helsinki.

For CD163 and Ki67 staining, double stains were performed sequentially on a Leica Bond™ instrument using the Bond Polymer Refine Detection System and the Bond Polymer Refine Red

Detection System.

Heat-induced epitope retrieval was done for 20 minutes with ER1 solution (Leica Microsystems AR9961). Ki67 (Clone MIB-1; DakoM7240) was used at a 1:20 dilution. CD163 (Clone 10D6; Leica NCL-CD163) was used at a 1:50 dilution. For quantification of CD163 positive cells, CD163 positive cells were counted in 10 randomly selected fields (×600 magnifications) for each tumor sample. Two independent investigators evaluated sections respectively.

Animal Studies

All studies were conducted under IACUC guidelines. 7 weeks old BALB/c female nude mice (National Cancer Institute) were injected subcutaneously with 1×10⁶1205Lu cells in 50% Matrigel (BD Biosciences) in both flanks of mice. When xenografts reached volumes of approximately 100 mm³, mice were randomly grouped to four groups, with 5 animals per group. GW2580 was dissolved in 0.5% hydroxypropylmethylcellulose (Sigma-Aldrich, MO, USA) and 0.1% Tween 80, and was dosed orally at 160 mg/Kg once daily. PLX4720 was dissolved 5% DMSO, 1% methylcellulose in distill water and was dosed orally at 30 mg/Kg twice a day. Tumor volumes were measured every three days using a digital caliber and were calculated using the equation V=0.5×L×W². Mice tumors were weighted after mice were euthanized.

For mouse tissues, formalin-fixed, paraffin-embedded mouse melanoma tumor tissues were deparaffinized, antigen retrieved as described previously. The tissues were then incubated with following antibodies: anti-Ki67 (Novus Biologicals), anti-F4/80 (Abcam), CD11b, CD31, phospho-ERK (Epitomics). After incubation with the primary antibody overnight at 4° C., a horseradish peroxidase (HRP)-conjugated Donkey anti-mouse or a Donkey anti-rabbit or a Donkey anti-rat IgG at a 1:200 dilution (Jackson ImmunoResearch) were used. Slides were subsequently incubated for 5 min in DAB (3,3′-diaminobenzidine) (Invitrogen) and counterstained with Haemalaun. For quantification of Ki67 positive cells, ki67 positive cells were counted in six fields (×400 magnifications) for each tumor sample (n=4 for each group). Two independent investigators evaluated sections respectively.

For flow cytometric analysis of peritoneal macrophages, 10 ml of cold PBS was intraperitoneally injected into the mice after mice were euthanized. Peritoneal cells were harvested and the numbers of macrophages were counted in a hemocytometer. Anti-mouse F4/80 and CD1b (BD Biosciences) were used to analyze the percent of macrophages by flow cytometric analysis.

Statistics

Paired two-tailed t-tests were performed to compare the difference in cell growth measurements between two samples. One-way analysis of variance (ANOVA) was used to examine the difference in tumor volumes at the end of the experiment among treatment groups. Two-way ANOVA was used to determine the effect of treatment groups with multiple concentrations of inhibitors.

The following examples elucidate how BRAF inhibition elicits profound effects on both tumor cells and macrophages, and suggests that the paradoxical activation of pathway in tumor stromal cells is one of major mechanisms for cancer cells to acquire resistance to target therapy.

### Example 14

### Macrophages are Essential for Melanoma Cell Growth and Survival Under BRAF Inhibition

To produce a model system that resembles the tumor microenvironment, the inventors co-cultured melanoma cells with human macrophages in a transwell co-culture system (11). The co-cultured cells were then exposed to BRAFi. Mutant BRAF^{V600E} melanoma cells, including 1205Lu, A375, SK-MEL-28 and 451 Lu, when cultured alone are sensitive to BRAFi.

1205Lu and A375 cells, and in a separate experiment, SK-MEL-28 and 451 Lu cells, were co-cultured in the presence or absence of macrophages with 0, 0.1, 1, 3 and 10 μM concentrations of an analog of the clinically approved vemurafenib, PLX4720, for 3 day. Cell growth was determined using WST-1 assay. Relative growth was calculated as the ratio of treated cells to untreated cells (without macrophage co-culture) at each dose. Melanoma cells were harvested and cell death was determined by flow cytometry using Annexin V and 7-AAD staining.

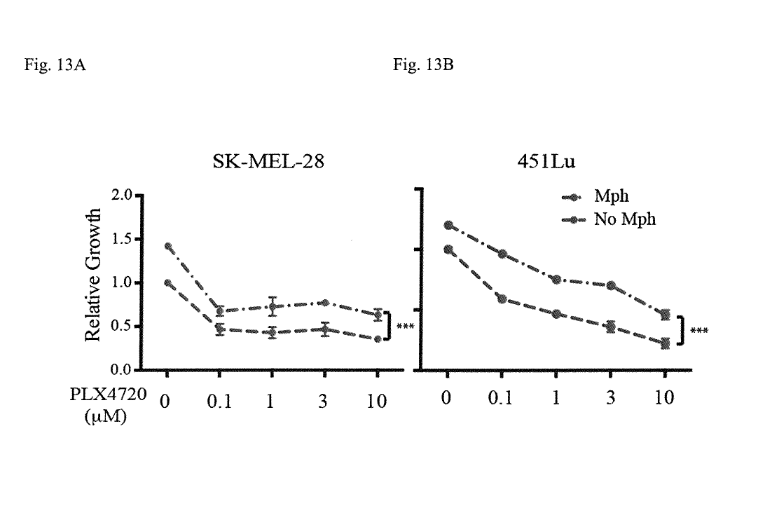

When co-cultured with macrophages and exposed to PLX4720, melanoma cells were significantly protected from PLX4720-induced growth inhibition and cell death, including apoptosis (Annexin V positive, 7-AAD negative) and necrosis (Annexin V and 7-AAD positive) (FIG. 9A, 9B, FIG. 13A, 13B). Macrophages activate p-ERK, but not p-AKT signaling in melanoma cells when PLX4720 was present (FIG. 9C; FIG. 2). Univariate Cox regression analysis showed statistically significant association between the number of melanoma-infiltrating macrophages with progression-free survival among 10 patients treated with BRAFi (FIGS. 9D, 9E).

Cell cycle analysis, performed by staining the 1205Lu and A375 cells with propidium iodide and analysis by flow cytometry (data not shown) confirmed that the percentage of the Sub-G population (apoptotic and necrotic cells) was significantly lower in the presence than absence of macrophages.

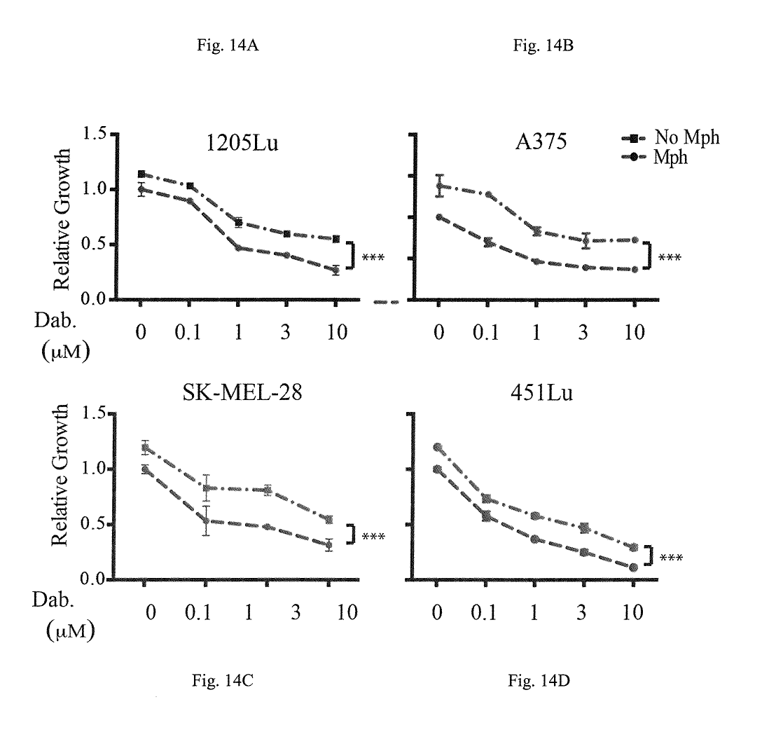

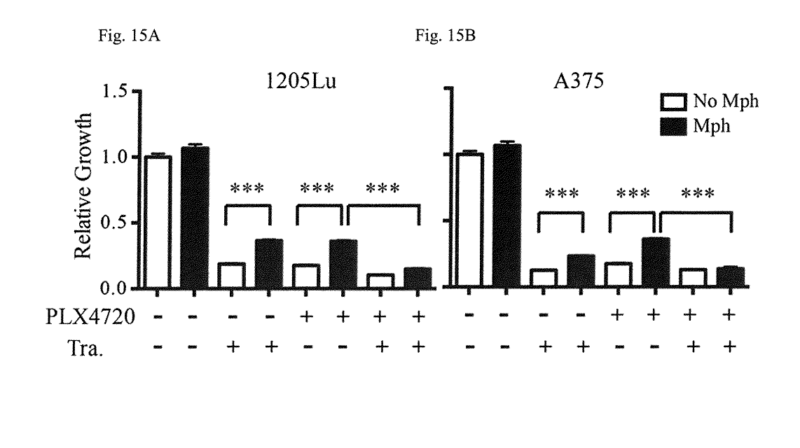

As further evidence that macrophages confer melanoma resistance to a BRAFi, 1205Lu, A375, SK-MEL-28, and 451Lu melanoma cells were co-cultured with or without macrophages in the presence of 0, 0.1, 1, 3 and 10 μM concentrations of a different BRAF inhibitor, Dabrafenib for 3 days. Similar results were obtained as for PLX4720 (FIGS. 14A-14D). Cell growth was measured by WST-1 assay Cells were harvested and cell death was determined by flow cytometry using Annexin V and 7-AAD staining with similar results (data not shown).

### Example 15

### Macrophage-Derived VEGF Confers Melanoma Resistance to BRAFi

1205Lu and A375 cells were cultured without or with VEGF (10 ng/ml) in the presence of PLX4720 for 3 days. DMSO was used as a control Cell growth was determined by WST-1 assay. Relative cell growth and cell death were determined as described above in Example 14. VEGF increases the activation of MAPK pathway. VEGF is shown to rescue PLX4720-induced melanoma growth inhibition and cell death in the presence of PLX4720. See FIGS. 10A, 10B.

1205Lu and A375 cells were cultured in the presence of VEGF (10 ng/ml) and PLX4720 (1 μM) for 4 hours. Cells were harvested for immunoblotting with antibodies to pERK, ERK, HSP90. VEGF increases the activation of MAPK pathway (FIG. 10C).

1205Lu and A375 cells were in the presence or absence of macrophages with PLX4720 (3 μM), anti-VEGF antibody (5 μg/ml), or both for 3 days, cell growth was determined by WST-1 assay. Relative cell growth and cell death were determined as above. Anti-VEGF reversed macrophage-mediated activation of the MAPK pathway and macrophage-mediated melanoma resistance to PLX4720 (FIGS. 10D, 10E, 10F).

### Example 16

### BRAF Inhibition Paradoxically Activates MAPK Pathway to Elicit Potent Biological Responses in Macrophages

BRAF inhibition activates MAPK pathway in macrophages as shown by the following data:

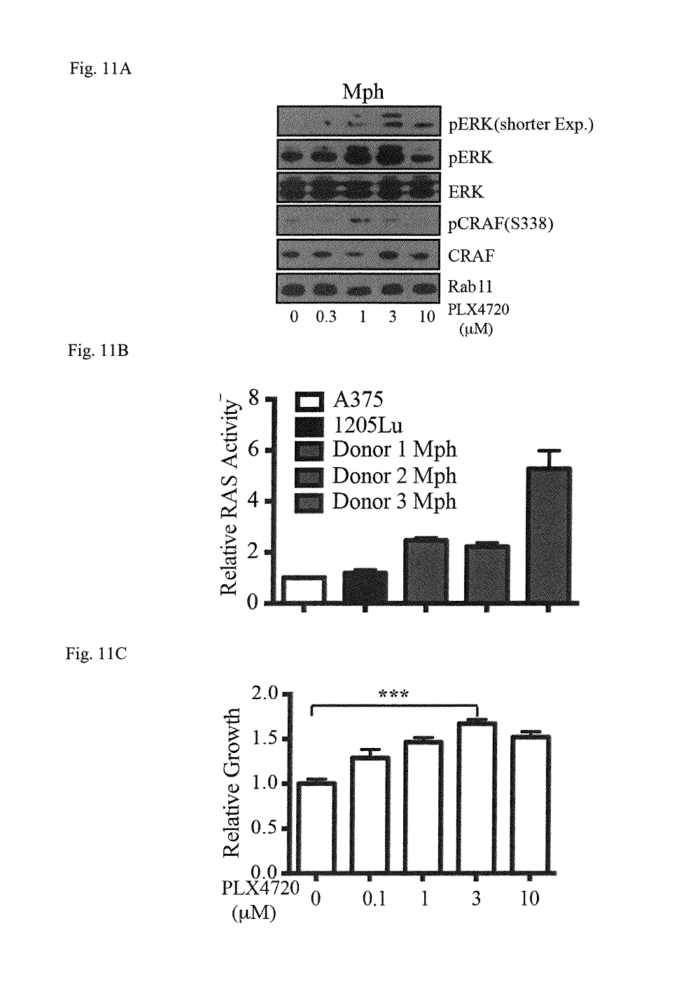

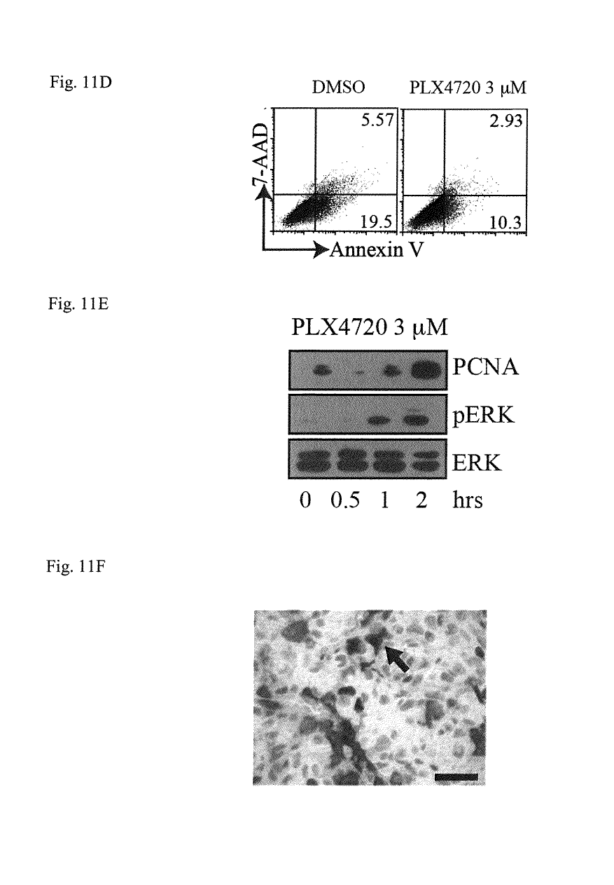

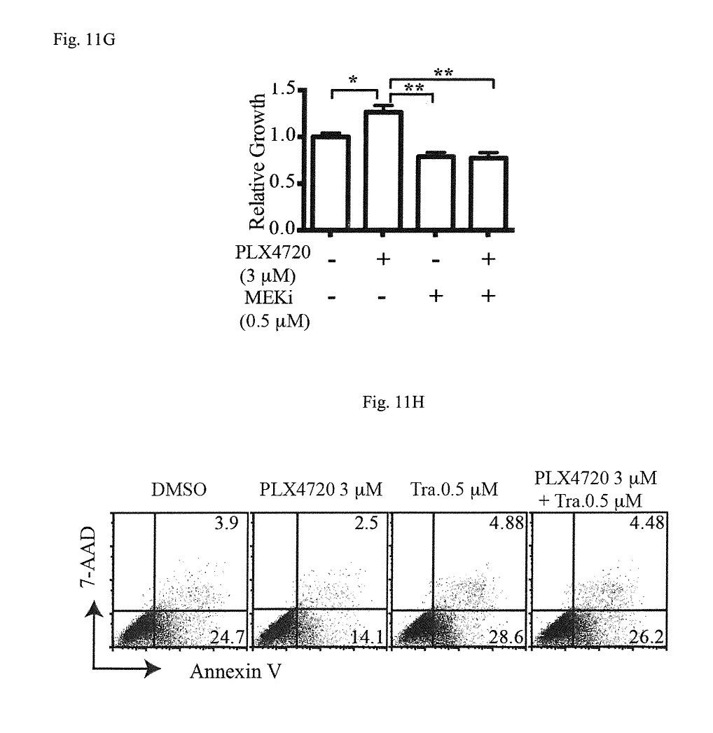

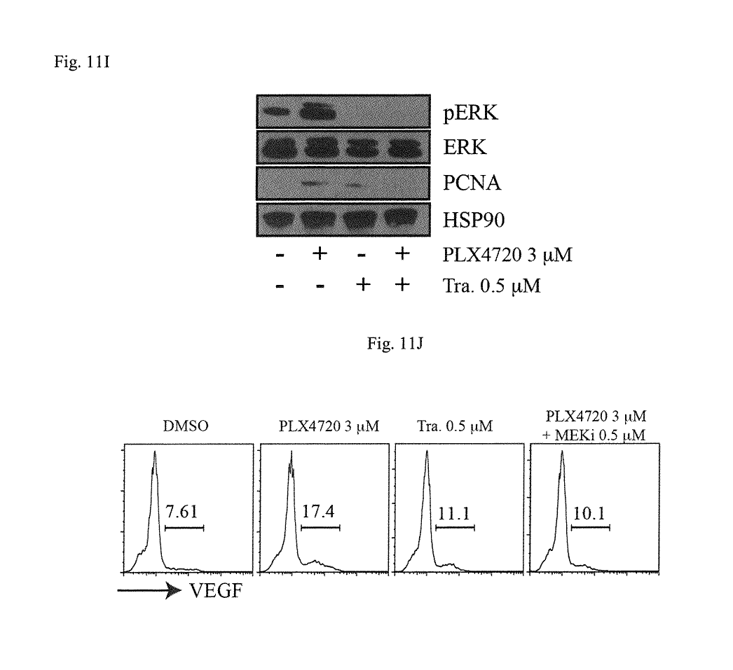

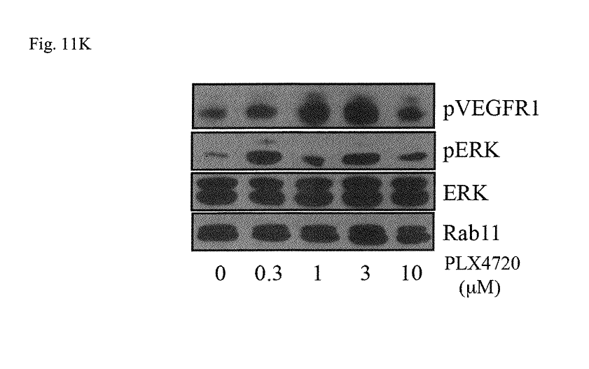

Macrophages were treated with 0, 0.3, 1, 3, and 10 μM concentration of PLX4720 for 2 hours. Cells were harvested for immunoblotting of antibodies to pERK (short expression), pERK, ERK, pCRAF (S338), CRAF, and Rab11 as shown in FIG. 11A. An ELISA assay was performed to determine the activation of RAS in 3 macrophage donors and the melanoma cells A375 and 1205Lu. As see in FIG. 11B, macrophages have high basal level of RAS activation. Cell lysates were harvested for immunoblotting of antibodies to pVEGFR1, pERK, ERK, Rab11, as seen in FIG. 11K.

Reactivation of the MAPK pathway and activation of alternative survival pathways, such as PI3K/AKT pathway are major mechanisms for melanoma cell resistance to BRAF inhibitors¹²⁻¹⁷. In yet another experiment in which melanoma cells in the presence of macrophages were treated with 0, 0.3, 1, 3, and 10 μM concentration of PLX4720 for 72 hours and cell growth was determined by WST-1 assay as in Example 14, BRAF inhibition is seen to promote macrophage growth (FIG. 11C). The presence of macrophages in co-culture system resulted in strong induction of ERK phosphorylation, but not other important melanoma signaling components such as AKT, NF-κB, CRAF and ARAF in melanoma cells (FIG. 11C). When 1205Lu cells were co-cultured with or without macrophages in the presence of PLX4720 (1 μM) for 6 hours and cell lysates were harvested for immunoblotting by antibodies against the following proteins, the macrophages do not activate CRAF, ARAF and NF-κB signaling (data not shown).

When macrophages were treated with 3 μM PLX4720 for 72 hours, harvested and cell death was determined as above (FIG. 11D), BRAF inhibition is seen to protect macrophage apoptosis.

BRAF inhibition was also seen to increase expression of PCNA (FIG. 11E).

MEK inhibitor Trametinib (tra.) was observed to reverse PLX4720-induced macrophage proliferation when macrophages were cultured in the presence of 3 μM concentrations of PLX4720 and 0.5 μM Trametinib or both for 3 days. Cell growth was determined by WST-1 assay as described above (FIG. 11G). FIG. 11H shows the results when the macrophages were treated above and cell death was analyzed by flow cytometry.

BRAF inhibition mediated ERK activation in macrophages was also shown to be reversed by MEK inhibition when macrophages were stimulated with 1 μM PLX4720 (PLX) or/and 0.5 μM Trametinib for 2 hours and cell lysates were harvested for immunoblotting of indicated antibodies (FIG. 11I). Additionally when 1205Lu Mph were treated with PLX4720, Trametinib or both, and incubated for 4 hours, subsequent intracellular staining performed to measure expression of VEGF showed that BRAF induced VEGF production (FIG. 11J).

In another experiment, 1205Lu and A375 melanoma cells were co-cultured with or without macrophages in the presence of indicated concentrations of PLX4720, Dab., Trametinib (Tra.), or combinations for 3 days. Cell growth was determined by WST-1 assay. Cells were treated as in a. and cell death was determined by flow cytometry using Annexin V and 7-AAD staining (data not shown). Macrophage-mediated resistance can be partially suppressed or reversed by a MEK inhibitor, Trametinib (FIGS. 15A, 15B). These data indicate that macrophage-mediated BRAFi resistance is due mostly to reactivation of the MAPK pathway, but other pathways may also contribute to macrophage-mediated BRAFi resistance, including PI3/AKT/mTOR pathways (FIG. 9C, right panel).

### Example 17

### Clinical Relevance of Tam

To examine the clinical relevance of the tumor associated-macrophages in melanomas on the anti-tumor responses to BRAFi, we co-stained pretreatment melanoma tissues from a panel of Stage IV melanoma patients treated with BRAFi with a proliferation marker Ki67, and a macrophage marker CD163. Tissue samples had been archived from 10 patients with stage IV melanoma treated with BRAFi. Immunohistochemistry analysis revealed that macrophages were abundant, and Ki67 positive melanoma cells were usually surrounded by macrophages, suggesting a microenvironment in which macrophages could provide growth stimulation for melanoma cells (FIGS. 9D and 11F). The specificity of anti-Ki67 and anti-CD163 antibodies was confirmed in human lymph node and placental tissues (data not shown).

Cox regression analysis was then used to examine the association between pre-treatment macrophage numbers and progression free survival. Patients with a higher number of pre-treatment macrophages were more likely to have worse progression-free survival hazards (ratio=1.138, p=0.046) (FIG. 9E).

### Example 18

### Mechanisms by which Growth Factors Confer Macrophage-Mediated Resistance

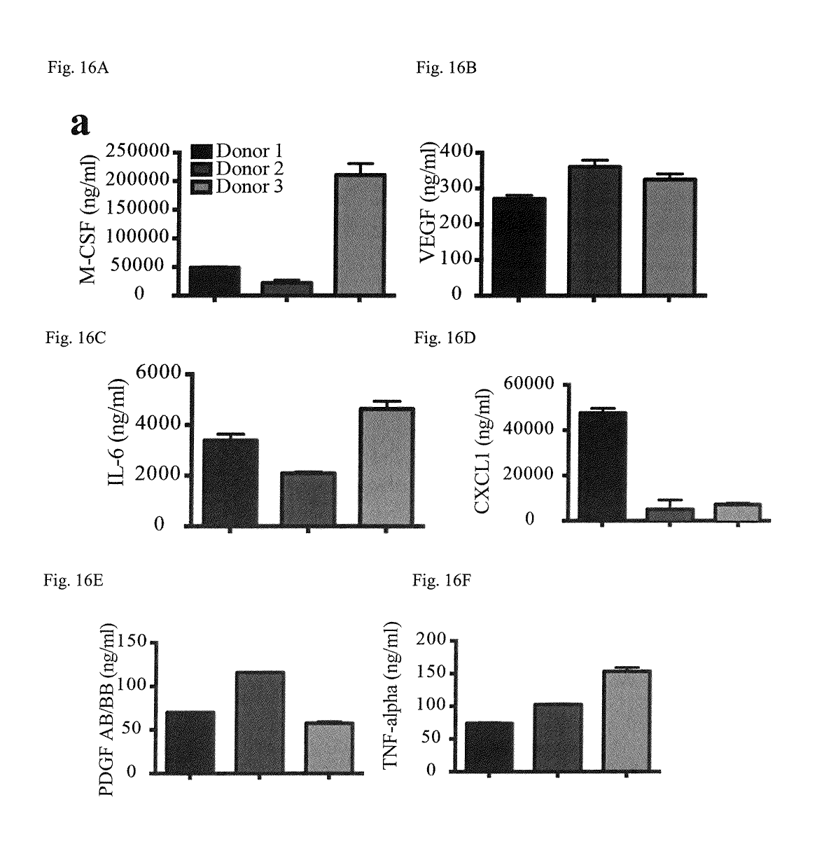

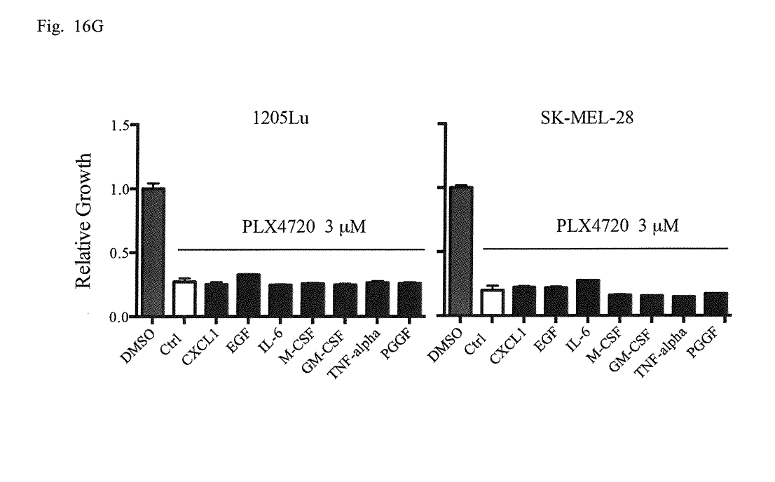

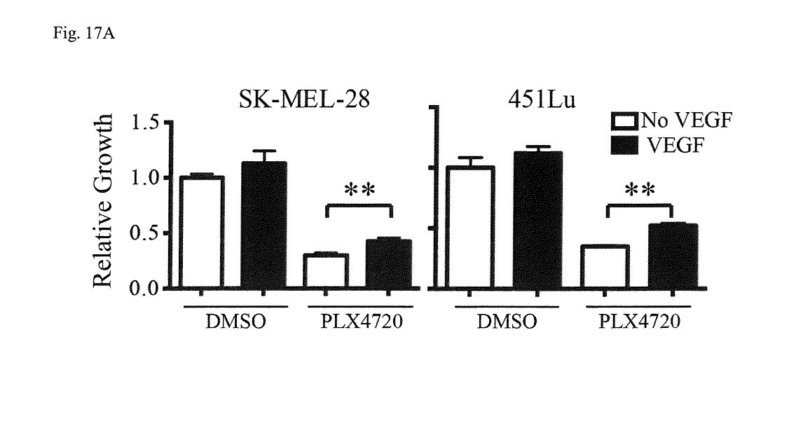

Many factors have been reported to rescue BRAFi-induced cell growth inhibition, including epidermal growth factor (EGF), fibroblast growth factor (FGF), and hepatocyte growth factor (HGF)¹⁸⁻²⁰. In addition, macrophages produce other factors that can also activate the MAPK pathway, including IL-6, M-CSF, CXCL1, GM-CSF, TNF-α, platelet-derived growth factor (PDGF), and VEGF (FIGS. 16A-16F).

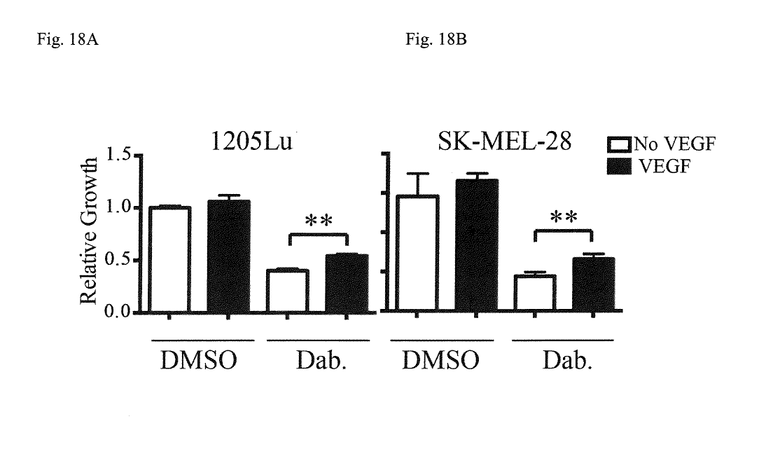

We found that only VEGF rescued PLX4720-induced cell growth inhibition (FIG. 10A, 10B, FIG. 16G, FIGS. 17A, 17B) and activated the MAPK pathway in melanoma cells (FIG. 11C). Similar effects were observed in Dabrafenib-treated melanoma cells (FIGS. 18A, 18B). HGF has been reported to have a similar effect but is mainly produced by fibroblasts and is present in the stromal component of patient tissues, which diminish its possible role in macrophage-mediated resistance (²⁰, data not shown).

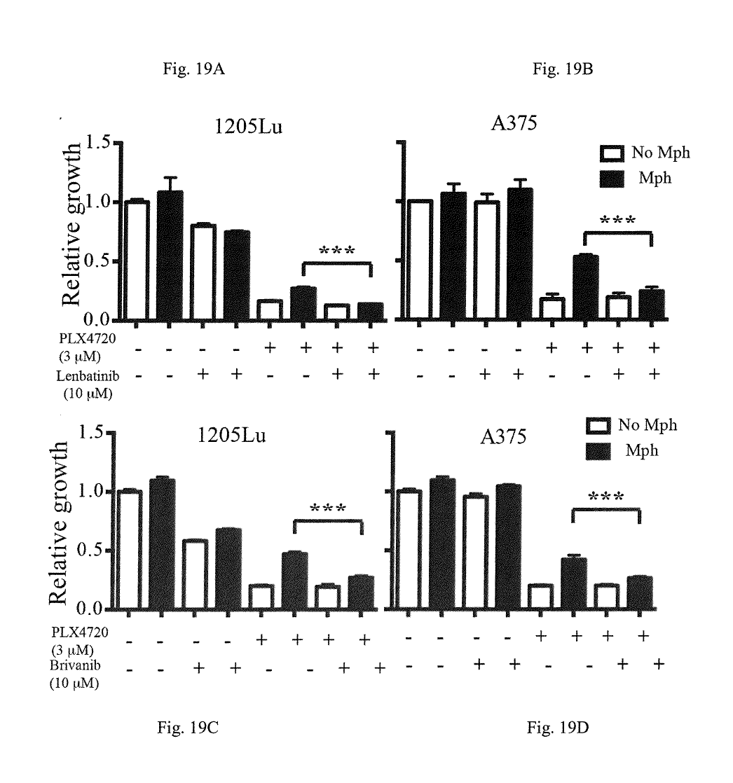

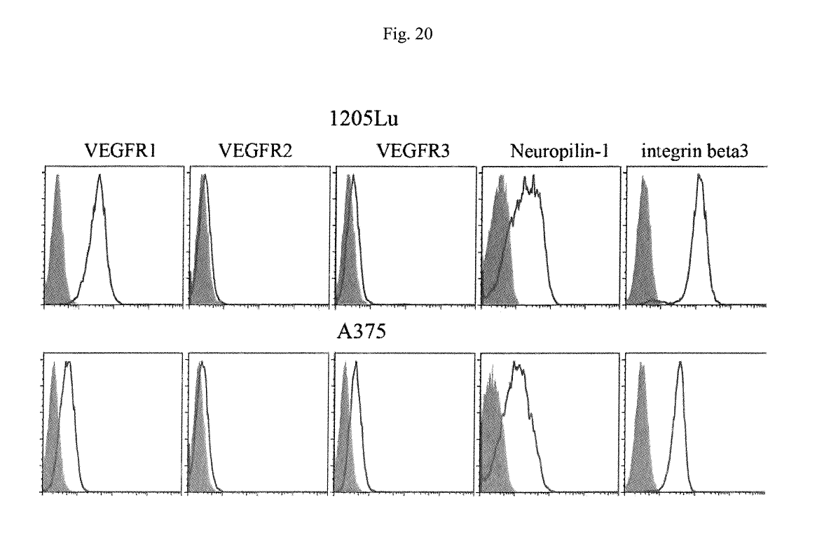

Blockade of VEGF signaling with an anti-VEGF monoclonal antibody significantly reversed macrophage-associated cell growth, and anti-cell death effect (FIG. 10D, 10E), as well as partially blocked reactivation of ERK signaling (FIG. 10F). Similar effects were observed using two VEGF receptor inhibitors (FIGS. 19A-19D). In addition to its angiogenic effect, VEGF has direct roles on tumor cells, including melanomas²¹⁻²⁵. VEGFR2, a major receptor for VEGF is expressed by melanoma cell lines and primary patient melanomas, and we also found that melanoma cells express all of the other co-receptors of VEGF (FIG. 20), providing a direct mechanistic link that explain how macrophage-derived VEGF can have direct effects on melanoma cells, especially when melanoma cells are under therapeutic stress.

### Example 19

### The Effect of Macrophages on Melanomas Grown in a Human Xenograft Model Treated with BRAFi

1205Lu cells were injected s.c into both flanks of nude mice. When the average tumor volume reached approximately 100 mm³, the doses of GW2580, (160 mg/Kg), PLX4720 (25 mg/Kg) or vehicle (n=10 for all groups, error bars indicate standard error) were administrated orally for 14 days. ANOVA was used to compare the differences in tumor volume. Mice were euthanized on day 14, peritoneal cells were harvested for flow cytometric analysis of the percent of F4/80 positive macrophages. Immunohistochemistry analysis of the expression of F4/80, phospho-ERK and Ki67 in tumors was conducted. The results are shown in FIGS. 12A-E. See, also the data in FIGS. 3B and 3C

A schematic model showing macrophages switching their roles from passenger to driver for melanoma growth and survival under BRAF inhibition is shown in FIG. 12F

After 14 days of treatment, GW2580, a small-molecule, ATP-competitive inhibitor of M-CSFR kinase (160 mg/kg), significantly decreased tumor size as a single therapeutic agent. It was less efficacious than PLX4720 (30 mg/ml) alone. A combination of both agents synergistically inhibited tumor growth and reduced tumor weight (FIG. 12A, FIG. 22A). The inhibitory effect of GW2580 on tumor growth is likely due to targeting macrophages, and not tumor cells directly, since GW2580 treatment can reverse macrophage-mediated resistance (FIG. 23A), and did not have significant effects on melanoma growth and death in vitro (FIG. 23B and data not shown). Consistent with previous studies, GW2580 treatment resulted in a significant decrease of the numbers of peritoneal F4/80 positive macrophages. PLX4720 treatment amplified this effect, though the mechanisms of this remains to be investigated (FIG. 12B, FIG. 23B). GW2580 treatment abolished F4/80 positive macrophages in tumors (FIG. 12C). Unlike human tumor-infiltrating macrophages, mouse tumor-infiltrating macrophages are mainly located around tumor blood vessel or necrotic tumor cells, which is consistent with previous studies. This may partially explain why PLX4720 treatment also results in significant decrease in the number of F4/80⁺ macrophages, because of its effect on angiogenesis by reducing macrophages migrating from blood to tumor tissues (data not shown). Similar results were obtained using another macrophage marker, CD11b (data not shown). There was decreased signaling of phospho-ERK and fewer Ki67 positive cells in tumor tissues treated with either GW2580 or PLX4720 or the combination of both compared to control mice (FIG. 12D, FIG. 22C). Toxicity was not detected in the therapy groups and all treated mice had similar body weight after treatment (FIG. 22D). Our data indicate that targeting macrophages alone can inhibit tumor growth and can increase the efficacy of BRAF inhibitors, which provides a rationale to combine BRAF inhibitors with therapies that target macrophages.

### Summary of Data in Example 13-19:

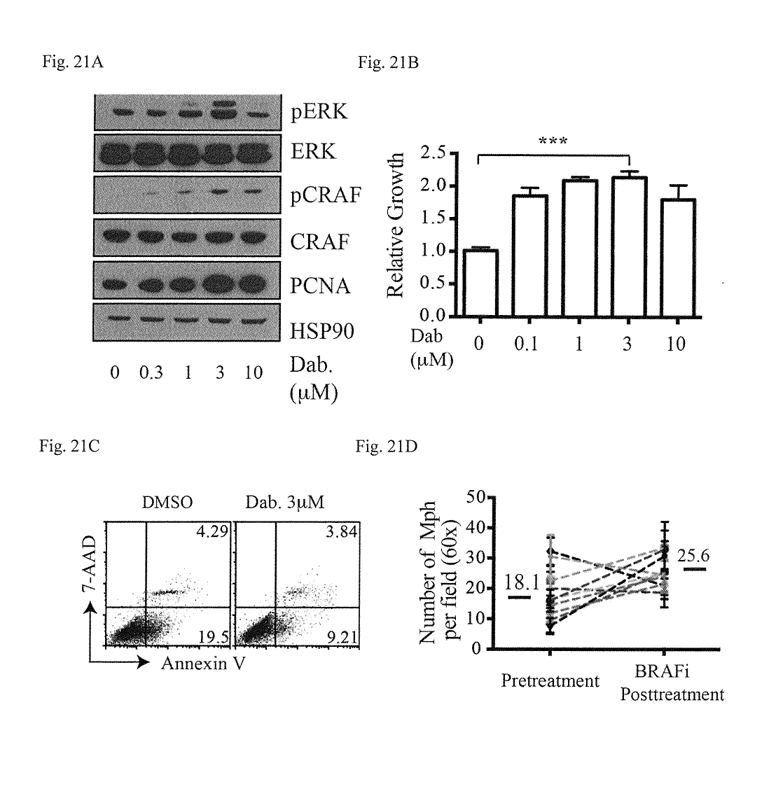

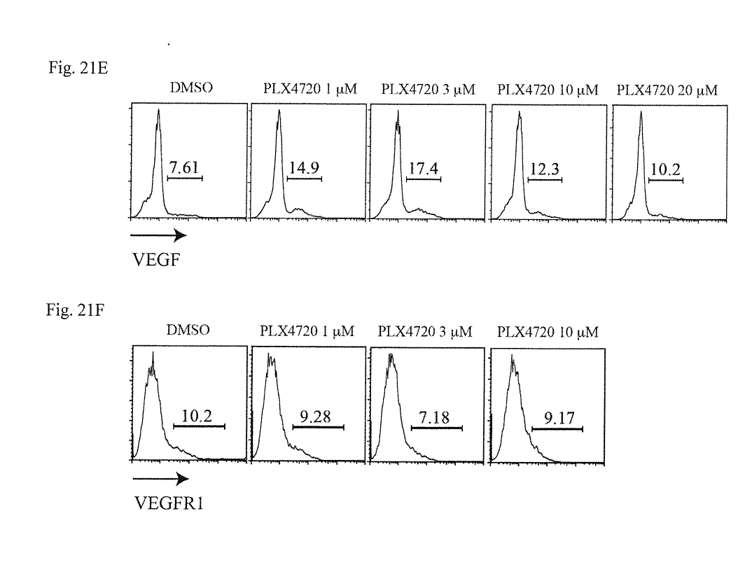

This data demonstrates that BRAF inhibition paradoxically activates the MAPK pathway in BRAF^{V600E} wild-type tumor cells via a RAS-dependent, CRAF activation mechanism²⁶⁻²⁹, or in activated RAS transfected keratinocytes. To more clearly understand the signaling pathways and biological consequence of BRAF inhibition on non-tumor cells, we investigated the effect of BRAF inhibition on signaling pathways and functions of a non-malignant cell, macrophages. Unlike previous reports showing PLX4720 only weakly activates the MAPK pathway in RAS mutant²⁹, BRAF wild-type cancer cells, PLX4720 strongly activated MAPK signaling in macrophages, accompanied by phosphorylation of CRAF (FIG. 11A). We observed a similar effect with Dabrafenib (FIGS. 21A-21F). Activation of the MAPK pathway by BRAFi requires a high level of activation of RAS, and we hypothesized that, like BRAF wild-type cancer cells, macrophages may have high basal level of endogenous RAS activation to activate MAPK upon BRAFi treatment. We confirmed by ELISA assay the high level of endogenous RAS activity in macrophages (FIG. 11B). By comparison, activation of the MAPK pathway in keratinocytes by BRAFi occurs only in mutant activated RAS transfected cells²⁶.

Functionally, BRAF inhibition stimulates macrophage growth and protects macrophage from cell death (FIG. 11C, 11D, FIG. 21B, 21C), as well as increases expression of the proliferation marker PCNA (FIG. 11E). Supporting this, immunohistochemistry analysis demonstrated the presence of Ki67-positive macrophages in BRAFi-treated patient tissues (FIG. 11F). Analysis of both pretreatment and posttreatment melanomas from ten patients treated with BRAFi indicated that more macrophages are present in tumors post-treatment (FIG. 21D). This effect may be due to BRAF inhibition, though we cannot exclude the attraction of macrophages to necrotic products following therapy (data not shown).

We then examined whether blockade of the MAPK pathway can reverse BRAFi-induced macrophage responses. The MEKi Trametinib inhibited PLX4720-induced proliferation and anti-cell death effects (FIG. 11G, 11H) and abolished PLX4720-induced ERK activation and PCNA expression (FIG. 11I). It has been reported that production of VEGF is induced by the activation of the MAPK pathway in endothelial cells. Flow cytometric analysis with intracellular staining for VEGF indicated that PLX4720 significantly increased the production of VEGF in macrophages, and blockade of MAPK activation by Trametinib abolished these effects (FIG. 11H; FIG. 21F). Moreover, BRAF inhibition also activated VEGFR1, indicating BRAF inhibition also exerts a paracrine effect on macrophages triggered by VEGF (FIG. 11I, data not shown). Together, our data indicate that BRAF inhibition elicits potent macrophage responses and increases the numbers of macrophages, as well as the production of VEGF, which then creates a potent stimulant for both macrophages and melanoma cells. Our data for the first time indicate that paradoxical activation of pathway by targeted therapies occurs in non-tumor cells, not in tumor cells or oncogene “primed” non-tumor cells. Importantly, the paradoxical activation of signaling pathways by specific small molecule inhibitors has been reported in other inhibitors and tumor types, suggesting our finding may also apply broadly for targeted therapies³⁰.

The data presented in the Examples herein suggests that without therapy, macrophages can provide survival signaling for melanoma cells, as evidenced by targeting macrophage alone can inhibit melanoma growth (FIG. 12A), but this has a moderate effect. Likely this is due to the many survival signaling pathways that are active in melanomas, which may only partially depend on stromal cells. Therefore, macrophages generally play a role as a passenger (FIG. 12E, left panel). When melanoma cells were exposed to BRAF inhibition, their growth pathways are interrupted, as evidenced by lower activity of ERK signaling (FIG. 10C) and they are more dependent on the survival signaling from macrophages. Importantly, macrophages also respond dynamically to BRAF inhibition to produce more growth factors such as VEGF, and are indispensible for melanoma cell growth and survival (FIG. 11C) resulting in a switch from a passenger to a driver (FIG. 12E, right panel). Our data demonstrate that macrophages also confer melanoma resistance to MEKi and the combination of BRAFi and MEKi, indicating the broad effects of macrophages on targeted therapy, though with different mechanisms (FIG. 11I, FIG. 15A, 15B). Our data may also explain why the combination of BRAFi and MEKi has better clinical efficacy, albeit resistance also develops.

This data changes the current paradigm of focusing on driver mutations in tumors to also considering other cells in the tumor environment as targets for anti-cancer therapies. Targeting macrophages, or in general, the tumor microenvironment along with therapies that target tumors should be considered an essential part of “cocktails” for melanoma therapy.

Each and every patent, patent application, including particularly U.S. provisional application No. 61/660,262 and the publication T. Wang et al, 2012 “Melanama-derived conditioned media efficiently induce the differentiation of monocytes to macrophages that display a highly invasive gene signature”, Pigment Cell Melanoma Res., 25(4):493-505; published on-line 9 Apr. 2012, doi: 10.1111/j.1755-148X.2012001005.x, and any document listed therein under References, and listed herein, and the sequence of any publically available nucleic acid and/or peptide sequence cited throughout the disclosure, is expressly incorporated herein by reference in its entirety. Embodiments and variations of this invention other than those specifically disclosed above may be devised by others skilled in the art without departing from the true spirit and scope of the invention. The appended claims include such embodiments and equivalent variations.

TABLE 3
(Sequence Listing Free Text)
The following information is provided for sequences
containing free text under numeric identifier <223>.

| SEQ ID NO: | Free text |
|---|---|
| (containing free text) | under <223> |
| 1 | Primer |
| 2 | Primer |
| 3 | Primer |
| 4 | Primer |
| 5 | Primer |
| 6 | Primer |
| 7 | Primer |
| 8 | Primer |
| 9 | Primer |
| 10 | Primer |
| 11 | Primer |
| 12 | Primer |

### REFERENCES

1 Davies, H. et al. Mutations of the BRAF gene in human cancer. Nature 417, 949-954 (2002).
2 Bollag, G. et al. Clinical efficacy of a RAF inhibitor needs broad target blockade in BRAF-mutant melanoma. Nature 467, 596-599 (2010).
3 Chapman, P. B. et al. Improved survival with vemurafenib in melanoma with BRAF V600E mutation. The New England Journal of Medicine 364, 2507-2516 (2011).
4 Flaherty, K. T. et al. Inhibition of mutated, activated BRAF in metastatic melanoma. The New England Journal of Medicine 363, 809-819 (2010).
5 R. Kefford, H. A., M. P. Brown, M. Millward, J. R. Infante, G. V. Long, D. Ouellet, M. Curtis, P. F. Lebowitz, G. S. Falchook. Phase I/II study of GSK2118436, a selective inhibitor of oncogenic mutant BRAF kinase, in patients with metastatic melanoma and other solid tumors. J Clin Oncol 28, Abstr 8503 (2010).
6 Acharyya, S. et al. A CXCL1 Paracrine Network Links Cancer Chemoresistance and Metastasis. Cell 150, 165-178 (2012).
7 Coussens, L. M., Zitvogel, L. and Palucka, A. K. Neutralizing tumor-promoting chronic inflammation: a magic bullet? Science 339, 286-291 (2013).
8 David G. DeNardo et al. Leukocyte Complexity Predicts Breast Cancer Survival and Functionally Regulates Response to Chemotherapy. Cancer Discovery 1, 2-16 (2011).
9 Gocheva, V. et al. IL-4 induces cathepsin protease activity in tumor-associated macrophages to promote cancer growth and invasion. Genes & Development 24, 241-255, doi:10.1101/gad.1874010 (2010).
10 Qian, B. Z. & Pollard, J. W. Macrophage diversity enhances tumor progression and metastasis. Cell 141, 39-51, doi:10.1016/j.cell.2010.03.014 (2010).
11 Wang, T. et al. Melanoma-Derived Conditioned Media Efficiently Induce the Differentiation of Monocytes to Macrophages that Display a Highly Invasive Gene Signature. Pigment Cell Melanoma Res 25(4):493-505(2012).
12 Flaherty, K. T., Hodi, F. S. & Fisher, D. E. From genes to drugs: targeted strategies for melanoma. Nature Reviews. Cancer 12, 349-361 (2012).
13 Nazarian, R. et al. Melanomas acquire resistance to B-RAF(V600E) inhibition by RTK or N-RAS upregulation. Nature 468, 973-977 (2010).
14 Paraiso, K. H. et al. Recovery of phospho-ERK activity allows melanoma cells to escape from BRAF inhibitor therapy. Br J Cancer 102, 1724-1730 (2010).
15 Poulikakos, P. I. et al. RAF inhibitor resistance is mediated by dimerization of aberrantly spliced BRAF(V600E). Nature 480, 387-390 (2011).
16 Ribas, A. & Flaherty, K. T. BRAF targeted therapy changes the treatment paradigm in melanoma. Nat Rev Clin Oncol 8, 426-433 (2011).
17 Villanueva, J., Vultur, A. & Herlyn, M. Resistance to BRAF inhibitors: unraveling mechanisms and future treatment options. Cancer Res 71, 7137-7140 (2011).
18 Metzner, T. et al. Fibroblast growth factor receptors as therapeutic targets in human melanoma: synergism with BRAF inhibition. The Journal of Investigative Dermatology 131, 2087-2095.
19 Straussman, R. et al. Tumor micro-environment elicits innate resistance to RAF inhibitors through HGF secretion. Nature 487, 500-504, (2012).
20 Wilson, T. R. et al. Widespread potential for growth-factor-driven resistance to anticancer kinase inhibitors. Nature 487, 505-9 (2012).
21 Adamcic, U., Skowronski, K., Peters, C., Morrison, J. & Coomber, B. L. The effect of bevacizumab on human malignant melanoma cells with functional VEGF/VEGFR2 autocrine and intracrine signaling loops. Neoplasia 14, 612-623 (2012).
22 Bougatef, F. et al. EMMPRIN promotes melanoma cells malignant properties through a HIF-2alpha mediated up-regulation of VEGF-receptor-2. PloS one 5, e12265, (2010).
23 Graells, J. et al. Overproduction of VEGF concomitantly expressed with its receptors promotes growth and survival of melanoma cells through MAPK and PI3K signaling. The Journal of Investigative Dermatology 123, 1151-1161 (2004).
24 Martin, M. J., Hayward, R., Viros, A. & Marais, R. Metformin accelerates the growth of BRAF V600E-driven melanoma by upregulating VEGF-A. Cancer Discovery 2, 344-355 (2012).
25 Valcarcel, M. et al. Vascular endothelial growth factor regulates melanoma cell adhesion and growth in the bone marrow microenvironment via tumor cyclooxygenase-2. Journal of Translational Medicine 9, 142, (2011).
26 Su, F. et al. RAS mutations in cutaneous squamous-cell carcinomas in patients treated with BRAF inhibitors. The New England Journal of Medicine 366, 207-215 (2012).
27 Poulikakos, P. I., Zhang, C., Bollag, G., Shokat, K. M. & Rosen, N. RAF inhibitors transactivate RAF dimers and ERK signaling in cells with wild-type BRAF. Nature 464, 427-430 (2010).
28 Heidorn, S. J. et al. Kinase-dead BRAF and oncogenic RAS cooperate to drive tumor progression through CRAF. Cell 140, 209-221 (2010).
29 Hatzivassiliou, G. et al. RAF inhibitors prime wild-type RAF to activate the MAPK pathway and enhance growth. Nature 464, 431-435 (2010).
30 Okuzumi, T. et al. Inhibitor hijacking of Akt activation. Nature Chemical Biology 5, 484-493, (2009).
31. Facchetti, F et al, KP1 (CD68) staining of malignant melanomas, Histopathology, 19, 141-145 (1991)

1. A composition comprising an agent, ligand or compound that inhibits or down-regulates macrophage production or activity in the subject for use in the treatment of melanoma.

2. The composition according to claim 1, further comprising an optional anti-melanoma agent.

3. The composition according to claim 1, wherein the agent, ligand or compound is
(a) an agent that-blocks or down-regulates the nucleic acid or protein expression or activity, or the downstream pathway of CCL-2;
(b) an agent that blocks or down-regulates the nucleic acid or protein expression or activity, or the downstream pathway of a matrix metalloprotease (MMP);
(c) an agent that blocks or down-regulates the nucleic acid or protein expression or activity of VEGF;
(d) an agent that blocks or down-regulates the expression, activity or signaling of the MAPK pathway;
(e) an agent that blocks or down-regulates the expression, activity or signaling of the PI3K-AKT-mTOR pathway;
(f) an agent that blocks or down-regulates the expression or activity of M-CSFR kinase; or
(g) an agent that blocks a receptor on macrophages.

4. The composition according to claim 3, comprising an antibody that binds CCL-2, an antibody that binds an MMP, an antibody that binds VEGF or a VEGF receptor inhibitor, a MEK inhibitor, or a M-CSFR inhibitor.

5. The composition according to claim 4, wherein the MMP is MMP-9.

6. The composition according to claim 4, wherein the M-CSFR inhibitor is GW2580.

7. The composition according to claim 2, wherein the anti-melanoma therapeutic agent is a BRAF inhibitor.

8. (canceled)

9. The composition according to claim 2, comprising a synergistic combination of an anti-melanoma therapeutic agent, which is the BRAF inhibitor PLX4720 and an M-CSFR inhibitor, GW2580.

10. (canceled)

11. A method for treating melanoma in a mammalian subject comprising reducing, inhibiting or down-regulating macrophage production or activity in the subject by administering a composition of claim 1.

12. The method according to claim 11, comprising reducing, inhibiting or down-regulating macrophage production or activity in the microenvironment of a melanoma tumor in the subject or by non-tumor cells in the subject.

13. (canceled)

14. The method according to claim 11, wherein the reducing, inhibiting or down-regulation of macrophage production or activity further comprises one or more of:
(a) blocking or down-regulating the nucleic acid or protein expression or activity, or the downstream pathway of CCL-2;
(b) blocking or down-regulating the nucleic acid or protein expression or activity, or the downstream pathway of a matrix metalloprotease (MMP);
(c) blocking or down-regulating the nucleic acid or protein expression or activity of VEGF;
(d) blocking or down-regulating the expression, activity or signaling of the MAPK pathway;
(e) blocking or down-regulating the expression, activity or signaling of the PI3K-AKT-mTOR pathway;
(f) blocking or down-regulating the expression or activity of M-CSFR kinase; and
(g) blocking a receptor on macrophages.

15. The method according to claim 14, wherein step (a) comprises administering to the subject with an antibody that binds CCL-2 or wherein step (b) comprises treating the subject with an antibody that binds an MMP or wherein step (c) comprises treating the subject with an antibody that binds VEGF or a VEGF receptor inhibitor.

16-18. (canceled)

19. The method according to claim 15 comprising treating the subject with a MEK inhibitor or with a M-CSFR inhibitor.

20. The method according to claim 11, wherein the reducing, inhibiting or down-regulation of macrophage production or activity occurs before, simultaneously with, or after, administration to the subject of a therapeutic agent directed against the tumor.

21-25. (canceled)

26. The method according to claim 11, further comprising:
(a) treating the subject with BRAF mutant melanoma with a BRAF inhibitor; and
(b) down-regulating macrophage activity in the subject before, simultaneously with, or after treatment with the BRAF inhibitor, thereby improving clinical outcome in the subject.

27-28. (canceled)

29. A method of diagnosing melanoma or determining its clinical prognosis in a mammalian subject comprising:
(a) detecting or measuring a modulation of nucleic acid expression or activity or an increase in the protein expression or activity of one or more of the genes of Table 1 in a biological sample of the subject and determining the status of disease relative to a control; or
(b) detecting or measuring a change in the amount or level of nucleic acid expression or activity or protein expression or activity of one or more of the biomarkers VEGF, M-CSFR, GPMNB, M-CSF, and CCL-2 in a biological sample of the subject and determining the status of disease relative to a control; or
(c) measuring the amount or level of tumor-infiltrating macrophages or a biomarker of said macrophages or a product secreted from said macrophages in a biological sample of the subject and determining the status of the melanoma relative to a control.

30. (canceled)

31. A method of differentiating human monocytes to macrophages comprising:
culturing human monocytes in concentrated melanoma tumor cell derived conditioned media (MCM), wherein the modified MCM is produced by culturing melanoma cells in melanoma media supplemented with fetal bovine serum (FBS), concentrating the harvested medium; and adding concentrated MCM to a complete medium at a suitable ratio.

32-45. (canceled)

46. A method for determining the efficacy of targeted cancer therapy comprising:
administering to a mammalian subject in need thereof a therapeutic treatment directed at inhibiting a targeted signaling pathway that enhances growth of a cancer or tumor cell;
assaying a biological sample of the subject to determine if that pathway is paradoxically activated in non-tumor cells of the subject;
wherein activation of the pathway in non-tumor cells during the course of the therapeutic treatment indicates a lack of efficacy or negative side effect of the therapeutic treatment.

47. The method of claim 46, wherein activation of the targeted pathway is detected by measuring the expression or activity of a gene or protein in the pathway or produced by activation of the pathway, wherein the cancer is melanoma; the therapeutic treatment is BRAF inhibitors; the targeted pathway is the MAPK pathway; and the pathway gene or protein is ERK.

48-49. (canceled)
